(19) United States
(12) Patent Application Publication
(10) Pub. No.: US 2005/0287666 A1
(43) Pub. Date: Dec. 29, 2005

(54) Cell culture medium comprising transition metals or trace elements

(75) Inventor: Robert W. Kenerson, Tonawanda, NY (US)

Correspondence Address: STERNE, KESSLER, GOLDSTEIN & FOX PLLC, 1100 NEW YORK AVENUE, N.W., WASHINGTON, DC, 20005, US

(73) Assignee: Invitrogen Corporation

(21) Appl. No.: 11/167,917

(22) Filed: Jun. 28, 2005

Related U.S. Application Data
(60) Provisional application No. 60/583,403, filed on Jun. 29, 2004.

(51) Int. Cl. C12N 5/08

(52) U.S. Cl. 435/372; 435/404

(57) ABSTRACT

The present invention provides improved cell culture media, media supplements, methods of manufacture of said media and methods of culturing cells. Transition metals or trace elements are controlled in a manner to provide improved conditions for culturing cells. Also provided are methods for supplementing media to improve culture and the supplemented cell culture media.

### CROSS-REFERENCE TO RELATED APPLICATION

This application claims priority to provisional application Ser. No. 60/583,403, filed on Jun. 29, 2004.

### BACKGROUND OF THE INVENTION

1. Field of the Invention

The present invention relates to cell culture media, preferably eukaryotic cell culture media, more preferably mammalian cell culture medium, methods of culturing cells, methods of manufacturing media, and methods of supplementing media to improve culture.

2. Related Art

Cell culture media provide nutrients necessary to maintain and grow cells in a controlled, artificial and in vitro environment. Characteristics and formulations of cell culture media vary depending upon the particular cellular requirements and special requirements that may result from the task the culture is designed to perform, e.g., recombinant protein synthesis. Important parameters include osmolarity, pH, and nutrient compositions.

Cell culture media formulations have been well documented in the literature and a large number of media are commercially available. In early cell culture work, media formulations were based upon the chemical composition and physicochemical properties (e.g., osmolality, pH, etc.) of blood and were referred to as “physiological solutions” (Ringer, S., J. Physiol. 3:380-393 (1880); Waymouth, C., In: Cells and Tissues in Culture, Vol. 1, Academic Press, London, pp. 99-142 (1965); Waymouth, C., In vitro 6:109-127 (1970)). However, cells in different tissues of a mammalian body are exposed to different microenvironments with respect to oxygen/carbon dioxide partial pressure and concentrations of nutrients, vitamins, and trace elements; accordingly, successful or optimal in vitro culture of different cell types often requires use of different medium formulations. Typical components of cell culture media include amino acids, organic and inorganic salts, vitamins, trace metals, sugars, lipids and nucleic acids, the types and amounts of which may vary depending upon the particular requirements of a given cell or tissue type. Media formulations are often based on basal or known formulations that are modified according to the skills of the cell culturist.

Medium formulations have been used to cultivate a number of cell types including animal, plant and bacterial cells. Cultivated cells have many uses including the study of physiological processes and the production of useful biological substances. Examples of such useful products include monoclonal antibodies, hormones, growth factors, enzymes and the like. Such products have many commercial and therapeutic applications and, with the advent of recombinant DNA technology, cells can be engineered to produce large quantities of these products. Cultured cells are also routinely used for the isolation, identification and growth of viruses which can be used as vectors and/or vaccines. Thus, the ability to cultivate cells in vitro is not only important for the study of cell physiology, but is also necessary for the production of useful substances which may not otherwise be obtained by cost-effective means.

A major focus in the field of experimental hematology (one active area of cell culture) continues to be the identification of the most primitive, pluripotent stem cell. One approach has been to identify cell surface markers (such as CD antigens) on the surface of progenitor cells and to correlate these markers with stages of development or differentiation by the cells' ability to form colonies of differentiated cells in methylcellulose culture systems. CD antigen expression has been shown to be modulated during cellular differentiation (Sieff, C. et al., Blood 60:703 (1982)). Hematopoietic stem cells are CD34⁺ cells. That is, they express the CD34 surface marker. The most primitive known human progenitor cell, which has been characterized as CD34⁺/CD33⁻/CD38⁻, represents only 1 to 2% of all bone marrow cells (Civin, C. I. et al., J. Immunol. 133:157 (1984)). Hematopoeitic, and other adult stem cells from virtually any organ or system, such a neural, hepatic, pancreatic, cardiac, myotonic, pleural, osteocytic, are cells of interest whose culture may benefit from the present invention. Cells at different stages of differentiation are expected to have different minimal requirements for nutrients than cells of the same lineage, but more or less differentiated or differentiated along different pathways.

Among the various cell types that have been grown using in vitro cell culture media, of particular interest are cells derived from the epithelium. Epithelium lines the internal and external surfaces of the organs and glands of higher organisms. Because of this localization at the external interface between the environment and the organism (e.g., the skin) or at the internal interface between an organ and the interstitial space (e.g., the intestinal mucosal lining), the epithelium has a major role in the maintenance of homeostasis. The epithelium carries out this function, for example, by regulating transport and permeability of nutrients and wastes (Freshney, R. I., in: Culture of Epithelial Cells, Freshney, R. I., ed., New York: Wiley-Liss, pp. 1-23 (1992)). Per.C6 and HEK 293 cells are common epithelial cells of interest in research and also for synthesis of biomolecules.

The cells making up the epithelium are generically termed epithelial cells. These cells can be present in multiple layers as in the skin, or in a single layer as in the lung alveoli. As might be expected, the structure, function and physiology of epithelial cells are often tissue-specific. For example, the epidermal epithelial cells of the skin are organized as stratified squamous epithelium and are primarily involved in forming a protective barrier for the organism, while the secretory epithelial cells of many glands are often found in single layers of cuboidal cells that have a major role in producing secretory proteins and glycoproteins. Regardless of their location or function, however, epithelial cells are usually regenerative. That is, under normal conditions, or in response to injury or other activating stimulus, epithelial cells are capable of dividing or growing. This regenerative capacity has facilitated the in vitro manipulation of epithelial cells, to the point where a variety of primary epithelial cells and cell lines have been successfully cultivated in vitro (Freshney, Id.).

While the isolation and use of a variety of epithelial cells and epithelial cell lines have been reported in the literature, the human embryonic kidney cell line 293 (“293 cells”), which exhibits epithelial morphology, has proven particularly useful for studies of the expression of exogenous ligand receptors, production of viruses and expression of allogeneic and xenogeneic recombinant proteins. For example, U.S. Pat. No. 5,166,066 describes the construction of a stable 293 cell line comprising functional GABA receptors (that include a benzodiazepine binding site) that have proven useful in identification and screening of candidate psychoactive drugs. 293 cells have also been used to produce viruses such as natural and recombinant adenoviruses (Gamier, A., et al., Cytotechnol. 15:145-155 (1994); Bout, A., et al., Cancer Gene Therapy 3(6):S24, abs. P-52 (1996); Wang, J.-W., et al., Cancer Gene Therapy 3(6):S24, abs. P-53 (1996)), which can be used for vaccine production or construction of adenovirus vectors for recombinant protein expression. Finally, 293 cells have proven useful in large-scale production of a variety of recombinant human proteins (Berg, D. T., et al., BioTechniques 14(6):972-978 (1993); Peshwa, M. V., et al., Biotechnol. Bioeng. 41:179-187 (1993); Gamier, A., et al., Cytotechnol. 15:145-155 (1994)). However, 293 cells are known to suffer from a lag phenomenon, e.g., when the cells are cultured to a density at or exceeding 2-2.5×10⁶ cells/ml, the next passage will exhibit a reduced growth. Apparently at least one apoptosis pathway is activated that results in a reduced growth and cell density for several subsequent passages. For bioproduction, consistent high cell density is preferred because of its correlation with lower cost.

Cells loosely called fibroblasts have been isolated from many different tissues and are understood to be connective tissue cells. It is clearly possible to cultivate cell lines, such as these fibroblastic cells, from embryonic and adult tissues. Fibroblasts characteristically have a “spindle” appearance. Fibroblast-like cells have morphological characteristics typical of fibroblast cells. Under a light microscope the cells appear pointed and elongated (“spindle shaped”) when they grow as a monolayer on the surface of a culture vessel. Cell lines can be regarded as fibroblast or fibroblast-like after confirmation with appropriate markers, such as collagen, type I ((Freshney, R. I., in: Culture of Epithelial Cells, Freshney, R. I., ed., New York: Wiley-Liss, pp. 1-23 (1987)).

CHO cells have been classified as both epithelial and fibroblast cells derived from the Chinese hamster ovary. A cell line started from Chinese hamster ovary (CHO-K1) (Kao, F.-T. And Puck, T. T., Proc. Natl. Acad. Sci. USA 60:1275-1281 (1968) has been in culture for many years but its identity is still not confirmed. Many adaptations of CHO cells such as CHO cells adapted for suspension culture have been developed for specialized uses.

Per.C6™ cells are also popularly used for expression models and for protein and vaccine production. Per.C6 cells are adenovirus transformed human retina cells that exhibit many desired characteristics for production of biomolecules such as therapeutic proteins. Typically, vector and packaging cells have to be adapted to one another so that they have all the necessary elements for expression, but they do not have overlapping elements which lead to replication competent virus by recombination. Therefore, the sequences necessary for proper transcription of the packaging construct may be heterologous regulatory sequences derived from, for example, other human adenovirus (Ad) serotypes, non-human adenoviruses, other viruses like, but not limited to, SV40, hepatitis B virus (HBV), Rous Sarcoma Virus (RSV), cytomegalo virus (CMV), etc. or from higher eukaryotes such as mammals. In general, these sequences include a promoter, enhancer and polyadenylation sequences. PER.C6 is an example of a cell line devoid of sequence overlap between the packaging construct and the adenoviral vector (Fallaux et al., 1998). Recombinant viruses based on subgroup C adenoviruses such as Ad5 and Ad2 can be propagated efficiently on these packaging cells. Generation and propagation of adenoviruses from other serotypes, like subgroup B viruses, has proven to be more difficult on PER.C6 cells. However, as described in European patent application 00201738.2, recombinant viruses based on subgroup B virus Ad35 can be made by co-transfection of an expression construct containing the Ad35 early region-1 sequences (Ad35-E1). Furthermore, Ad35-based viruses that are deleted for E1A sequences were shown to replicate efficiently on PER.C6 cells. Thus, the E1A proteins of Ad5 complement Ad35-E1A functions, whereas at least part of the E1B functions of Ad35 are necessary. This serotype specificity in E1B functions was recently also described for Ad7 recombinant viruses. In an attempt to generate recombinant adenoviruses derived from subgroup B virus Ad7, Abrahamsen et al. (1997) were not able to generate E1-deleted viruses on 293 cells without contamination of wild-type (wt) Ad7. Viruses that were picked after plaque purification on 293-ORF6 cells (Brough et al., 1996) were shown to have incorporated Ad7 E1B sequences by non-homologous recombination. Thus, efficient propagation of Ad7 recombinant viruses proved possible only in the presence of Ad7-E1B expression and Ad5-E4-ORF6 expression. The E1B proteins are known to interact with cellular as well as viral proteins (Bridge et al., 1993; White, 1995). Possibly, the complex formed between the E1B 55K protein and E4-ORF6 which is necessary to increase MRNA export of viral proteins and to inhibit export of most cellular mRNAs, is critical and in some way serotype specific. Antibody production is one task that Per.C6 cells have been successfully cultured to achieve. PER.C6 has been deposited at the ECACC under number 96022940.

Most primary mammalian epithelial cells, mammalian fibroblast cells, epithelial cell lines, and fibroblast cell lines have been traditionally grown in monolayer culture. For some applications, however, it is advantageous to cultivate such cells as suspension cultures. For example, suspension cultures grow in a three-dimensional space. Monolayer cultures in similar-sized vessels, however, can only grow two-dimensionally on the vessel surface. Thus, suspension cultures can result in higher cell yields and, correspondingly, higher yields of biologicals or biomolecules (e.g., viruses, recombinant polypeptides, etc.) compared to monolayer cultures. In addition, suspension cultures are often easier to feed and scale-up, via simple addition of fresh culture media (dilution subculturing) to the culture vessel rather than trypsinization and centrifugation as is often required with monolayer cultures. The ease of feeding and the ease with which suspension cultures can be scaled up represent a substantial saving in time and labor for handling a comparable number of cells. Several cell lines, such as Per.C6 and suspension adapted CHO and 293 cell lines have been developed and studied to meet these perceived advantages.

Many anchorage-dependent cells, such as primary epithelial cells, primary fibroblast cells, epithelial cell lines, and fibroblast cell lines, however, are not easily adapted to suspension culture. Since they are typically dependent upon anchorage to a substrate for optimal growth, growth of these cells in suspension can require their attachment to microcarriers such as latex or collagen beads. Thus, cells grown in this fashion, while capable of higher density culture than traditional monolayer cultures, are still technically attached to a surface; subculturing of these cells therefore requires similar steps as those used for the subculturing of monolayer cultures. Furthermore, when large batch or fermenter cultures are established, a large volume of microcarriers often settles to the bottom of the culture vessel, thereby requiring a more complicated agitation mechanism to keep the microcarriers (and thus, the cells) in suspension without causing shear damage to the cells (Peshwa, M. V., et al., Biotechnol. Bioeng. 41:179-187 (1993)). Regardless of whether cells are in suspension, in monolayer culture or attached on microcarriers, the cells will have their minimal as well as optimal nutrient requirements. Thus the content of trace elements as one nutrient class is an important consideration for all cells regardless of the specific culture conditions.

Although many transformed cells are capable of being grown in suspension (Freshney, R. I., Culture of Animal Cells: A Manual of Basic Technique, New York: Alan R. Liss, Inc., pp. 123-125 (1983)), successful conventional suspension cultures often require relatively high-protein media or supplementation of the media with serum or serum components (such as the attachment factors fibronectin and/or vitronectin), plant animal or microorganism hydrolysates, or sophisticated perfusion culture control systems (Kyung, Y.-S., et al., Cytotechnol. 14:183-190 (1994)), which can be disadvantageous. In addition, many epithelial cells when grown in suspension form aggregates or “clumps” which can interfere with successful subculturing and reduce growth rate, successful harvesting and/or production of biologicals by the cultures. When clumping occurs, the overall cellular surface area exposed to medium is decreased and the cells are deprived of nutrition and are unable to efficiently exchange waste into the medium. As a result, growth slows, diminished cell densities are obtained, protein and other biomolecule expression is compromised, and yields of biomolecules suffer. Trace element balance is one factor that can be manipulated to control clump formation.

Typically, cell culture media formulations are supplemented with a range of additives, including undefined components such as fetal bovine serum (FBS) (5-20% v/v) or extracts from animal embryos, organs or glands (0.5-10% v/v). While FBS is the most commonly applied supplement in animal cell culture media, other serum sources are also routinely used, including newborn calf, horse and human. Organs or glands that have been used to prepare extracts for the supplementation of culture media include submaxillary gland (Cohen, S., J. Biol. Chem. 237:1555-1565 (1961)), pituitary (Peehl, D. M., and Ham, R. G., In vitro 16:516-525 (1980); U.S. Pat. No. 4,673,649), hypothalamus (Maciag, T., et al., Proc. Natl. Acad. Sci. USA 76:5674-5678 (1979); Gilchrest, B. A., et al., J. Cell. Physiol. 120:377-383 (1984)), ocular retina (Barretault, D., et al., Differentiation 18:29-42 (1981)) and brain (Maciag, T., et al., Science 211:1452-1454 (1981)). These types of chemically undefined supplements serve several useful functions in cell culture media (Lambert, K. J. et al., In: Animal Cell Biotechnology, Vol. 1, Spier, R. E. et al., Eds., Academic Press New York, pp. 85-122 (1985)). For example, these supplements provide carriers or chelators for labile or water-insoluble nutrients; bind and neutralize toxic moieties; provide hormones and growth factors, protease inhibitors and essential, often unidentified or undefined low molecular weight nutrients; and protect cells from physical stress and damage. Thus, serum or organ/gland extracts are commonly used as relatively low-cost supplements to provide an efficient culture medium for the cultivation of animal cells. As alluded to below, these undefined components often include transition elements and other salts in unknown and uncontrolled amounts. Modifying trace element content to improve batch consistency is thus advantageous in defined as well as undefined cultures.

Unfortunately, use of serum or organ/gland extracts in tissue culture applications has several drawbacks (Lambert, K. J. et al., In: Animal Cell Biotechnology, Vol. 1, Spier, R. E. et al., Eds., Academic Press New York, pp. 85-122 (1985)). For example, chemical compositions of these supplements and sera vary between lots, even from a single manufacturer. The supplements can also be contaminated with infectious agents (e.g., mycoplasma and viruses) which can seriously undermine the health of the cultured cells and the quality of the final product. The use of undefined components such as serum or animal extracts also prevents the true definition and elucidation of the nutritional and hormonal requirements of the cultured cells, thus eliminating the ability to study, in a controlled way, the effect of specific growth factors or nutrients on cell growth and differentiation in culture. Moreover, undefined supplements prevent the researcher from studying aberrant growth and differentiation and the disease-related changes in cultured cells. Finally and most importantly to those employing cell culture media in the industrial production of biological substances (e.g., biomolecules), serum and organ/gland extract supplementation of culture media can complicate and increase the costs of regulatory compliance and the purification of the desired substances from the culture media due to nonspecific co-purification of serum or extract proteins.

Improved levels of recombinant protein expression are obtained from cells grown in serum-free medium, relative to the level of expression seen in cells grown in medium supplemented with serum (Battista, P. J. et al., Am. Biotech. Lab. 12:64-68 (1994)). However, serum-free media can still contain one or more of a variety of animal-derived components, including albumin, fetuin, various hormones and other proteins. The presence of proteins or peptides makes purification of recombinant protein or other biomolecule difficult, time-consuming, and expensive. If non-protein substitutes could be found to replace these components final yields of biomolecules could be improved.

To overcome these drawbacks of the use of serum or organ/gland extracts, a number of so-called “defined” media have been developed. These media, which often are specifically formulated to support the culture of a single cell type, contain no undefined supplements and instead incorporate defined quantities of purified growth factors, proteins, lipoproteins and other substances usually provided by the serum or extract supplement. Since the components (and concentrations thereof) in such culture media are precisely known, these media are generally referred to as “defined culture media.” Sometimes used interchangeably with “defined culture media” is the term “serum-free media” or “SFM.” A number of SFM formulations are commercially available, such as those designed to support the culture of endothelial cells, keratinocytes, monocytes/macrophages, lymphocytes, hematopoietic stem cells, fibroblasts, chondrocytes or hepatocytes which are available from Invitrogen Corporation, Carlsbad, Calif. The distinction between SFM and defined media, however, is that SFM are media devoid of serum and protein fractions (e.g., serum albumin), but not necessarily of other undefined components such as organ/gland extracts. Indeed, several SFM that have been reported or that are available commercially contain such undefined components, including several formulations supporting in vitro culture of keratinocytes (Boyce, S. T., and Ham, R. G., J. Invest. Dermatol. 81:33 (1983); Wille, J. J., et al., J. Cell. Physiol. 121:31 (1984); Pittelkow, M. R., and Scott, R. E., Mayo Clin. Proc. 61:771 (1986); Pirisi, L., et al., J. Virol. 61:1061 (1987); Shipley, G. D., and Pittelkow, M. R., Arch. Dermatol. 123:1541 (1987); Shipley, G. D., et al., J. Cell. Physiol. 138:511-518 (1989); Daley, J. P., et al., FOCUS (GIBCO/LTI) 12:68 (1990); U.S. Pat. Nos. 4,673,649 and 4,940,666): SFM thus cannot be considered to be equivalent to defined media in the true definition of the term. While defined media have special needs, for example replacing the functions of unknown compounds, including trace elements, contributed by the undefined component, balancing of undefined ingredients may remove some of the batch to batch variability of the non-CD media.

Defined media generally provide several distinct advantages to the user. For example, the use of defined media facilitates the investigation of the effects of a specific growth factor or other medium component on cellular physiology, which can be masked when the cells are cultivated in serum- or extract-containing media. In addition, defined media typically contain much lower quantities of protein (indeed, defined media are often termed “low protein media”) than those containing serum or extracts, rendering purification of biological substances produced by cells cultured in defined media far simpler and less expensive. However, even in defined media a batch to batch variability can occur. Thus in order to minimize uncontrolled variables, researchers desire performing all experiments from a single lot number. However, as culture moves into bioproduction applications individual batches or lots may only ill the needs of a single bioreactor run. Consistency between batches thus takes on a greater significance.

Some extremely simple defined media, which consist essentially of vitamins, amino acids, organic and inorganic salts and buffers have been used for cell culture. Such media (often called “basal media”), however, are usually seriously deficient in the nutritional content required by most animal cells, especially specific nutrient requirements of specialized or differentiated cells. Accordingly, most defined media incorporate into the basal media additional components to make the media more nutritionally complex, but to maintain the serum-free and low protein content of the media. Examples of such components include bovine serum albumin (BSA) or human serum albumin (HSA); certain growth factors derived from natural (animal) or recombinant sources such as epidermal growth factor (EGF) or fibroblast growth factor (FGF); lipids such as fatty acids, sterols and phospholipids; lipid derivatives and complexes such as phosphoethanolamine, ethanolamine and lipoproteins; protein and steroid hormones such as insulin, hydrocortisone and progesterone; nucleotide precursors; and certain trace elements (reviewed by Waymouth, C., in: Cell Culture Methods for Molecular and Cell Biology, Vol. 1: Methods for Preparation of Media, Supplements, and Substrata for Serum-Free Animal Cell Culture, Barnes, D. W., et al., eds., New York: Alan R. Liss, Inc., pp. 23-68 (1984), and by Gospodarowicz, D., Id., at pp 69-86 (1984)).

The use of animal protein supplements in cell culture media, however, also has certain drawbacks. For example, there is a risk that the culture medium and/or products purified from it can be immunogenic, particularly if the supplements are derived from an animal different from the source of the cells to be cultured. Depending on specifics how the protein is manufactured and purified, there can be batch to batch variability in the biomolecule additives, such as ions or other biomolecules that copurify to a greater or lesser degree with the biomolecule of interest. If biological substances to be used as therapeutics are purified from such culture media, certain amounts of these immunogenic proteins or peptides can be co-purified and can induce an immunological reaction, up to and including anaphylaxis, in an animal receiving such therapeutics.

To obviate this potential problem, supplements derived from the same species as the cells to be cultured can be used. For example, culture of human cells can be facilitated using HSA as a supplement, while media for the culture of bovine cells would instead use BSA. This approach, however, runs the risks of introducing contaminants and adventitious pathogens into the culture medium (such as Creutzfeld-Jakob Disease (CJD) from HSA preparations, or Bovine Spongiform Encephalopathy (“Mad Cow Disease”) prion from BSA preparations), which can obviously negatively impact the use of such media in the preparation of animal and human therapeutics. In fact, for such safety reasons, the biotechnology industry and government agencies are increasingly regulating, discouraging, and even forbidding the use of cell culture media containing animal-derived proteins which can contain such pathogens. Even recombinant products will suffer from batch to batch inconsistency and may introduce adventitious agents or other problematic constituents that copurify with the biomolecule of interest.

To overcome the limitations of the use of animal proteins in SFM, several attempts have been made to construct animal cell culture media that are completely free of animal proteins. For example, some culture media have incorporated extracts of yeast cells into the basal medium (see, for example, U.K. Patent Application No. GB 901673; Keay, L., Biotechnol. Bioengin. 17:745-764 (1975)) to provide sources of nitrogen and other essential nutrients. In another approach, hydrolysates of wheat gluten have been used, with or without addition of yeast extract, to promote in vitro growth of animal cells (Japanese Patent Application No. JP 249579). Still other media have been developed in which serum is replaced by enzymatic digests of meat, or of proteins such as α-lactalbumin or casein (e.g., peptone), which have been traditionally used in bacterial culture (Lasfargues, E. Y., et al., In vitro 8(6):494-500 (1973); Keay, L., Biotechnol. Bioeng. 17:745-764 (1975); Keay, L., Biotechnol. Bioeng. 19:399-411 (1977); Schlager, E. J., J. Immunol. Meth. 194:191-199 (1996)). None of these approaches, however, provided a culture medium optimal for the cultivation of a variety of animal cells. Moreover, extracts from certain plants, including wheat, barley, rye and oats have been shown to inhibit protein synthesis in cell-free systems derived from animal cells (Coleman, W. H., and Roberts, W. K., Biochim. Biophys. Acta 696:239-244 (1982)), suggesting that the use of peptides derived from these plants in cell culture media can actually inhibit, rather than stimulate, the growth of animal cells in vitro. More recently, animal cell culture SFM formulations comprising rice peptides have been described and shown to be useful in cultivation of a variety of normal and transformed animal cells (see U.S. Pat. No. 6,103,529, incorporated herein by reference in its entirety). However, these undefined extracts are a potential source of adventitious agents or other variables and are always suspected when variability between media batches is encountered.

Notwithstanding the potential difficulties posed by addition of animal derived supplements to cell culture media, such supplements are in routine use. One such supplement that is frequently added to defined media is a metal carrier such as transferrin. Transferrin functions in vivo to deliver iron to cells. The mechanism of iron uptake by mammalian cells has been reviewed (Qian, Z. M. and Tang, P. L. (1995) Biochim. Biophys. Acta 1269, 205-214). As iron is required as a co-factor in numerous metabolic processes including energy generation and oxidative respiration, serum-free media are often supplemented with transferrin in order to deliver the requisite iron for the successful cultivation of most cells in vitro. Concern about various potential adventitious agents in preparations of transferrin has stimulated a search for other natural iron carrier compounds which can be used as a substitute for transferrin. This search is complicated by the fact that the natural iron carriers are often derived from serum and thus are subject to the above-described limitations of serum supplementation. Transferrin is known to bind iron and other trace elements. Depending on the precise binding conditions, including pH, temperature, ionic strength, etc., variability in trace element content is expected whenever, metal carriers for example synthetic or natural chelators, e.g., transferrin are present.

To overcome the limitations of using naturally derived metal carriers, certain metal binding compounds are being explored for use in supplying metals, particularly zinc, iron, manganese and magnesium, to cultured cells. Simple carriers such as chelating agents (e.g., EDTA) and certain acids or salts thereof (e.g., citrate, picolinate, and derivatives of benzoic acid or hydroxamic acid) have been shown to be useful in certain serum-free growth media (see U.S. Pat. Nos. 5,045,454 and 5,118,513; Testa et al., Brit. J. Haematol. 60:491-502, (1985); Ganeshaguru et al., Biochem. Pharmacol. 29:1275-1279 (1980); White et al., Blood 48:923-929 (1976)).

Although these references disclose some metal carriers, the interpretation of the data is complicated by several experimental factors. The data were gathered from a limited number of cell lines and show results of a single passage. In addition, the media were supplemented with serum. Serum inherently contains transferrin and other potential iron carriers. There is a “carry-over effect” on growth of cells which have been cultured in serum-supplemented medium, even after one or two passages in the absence of serum or transferrin (see, for example, Keenan, J. and Clynes, M. (1996) In vitro Cell Dev. Biol-Animal 32, 451-453). Other known metal binding compounds have been used medicinally to remove iron from the body and not for delivery. Unfortunately, many of these simple iron chelating compounds do not provide sufficient iron availability to, or uptake by, cultured cells and will carry other elements in trace quantities.

Once a suitable medium formulation for the growth of a particular cell type has been determined, based on cell requirements, purification requirements, cost, etc., it is frequently necessary to alter the cell in question so as to optimize the production of a desired biological substance. A critical step in the effective production and purification of biological substances is the introduction of one or more macromolecules (e.g., peptides, proteins, nucleic acids, etc.) into the cell in which the material will be produced. This can be accomplished by a variety of methods. One widely used method to introduce macromolecules into a cell is known as transfection.

Typically, the target cell is grown to a desired cell density in a cell culture medium optimized for growth of the cell. Once the desired density is reached, the medium is exchanged for a medium optimized for the transfection process. Under most circumstances, the medium used for transfection does not support the growth of the cells but the transfection medium is merely used for the purpose of introducing nucleic acids into the cells. As a result, the process generally requires collecting the cells from the culture, usually by centrifugation, washing the cells to remove traces of the growth medium, suspending the cells in a transfection medium in the presence of the macromolecule of interest, incubating the cells in the transfection medium for a period of time sufficient for the uptake of the macromolecule, optionally, removing the transfection medium and washing the remnants of the transfection medium from the cells and then re-suspending the transfected cells in a growth medium. The steps of exchanging growth media for transfection media, washing the cells, and exchanging the transfection medium back to a growth medium require a great deal of hands-on manipulation of the cells thereby adding substantially to the time and expense of recombinant DNA technology. Proper balance of trace elements may minimize the need for multiple media for different phases of bioproduction.

As an historical example, 293 cells have been cultivated in monolayer cultures in a serum-supplemented version of a complex medium (i.e., DMEM). When grown in suspension, 293 cells have a tendency to aggregate into large clusters of cells. The formation of these large cell aggregates reduces the viability of the cells. Since the cells in the center of the aggregates are not directly exposed to the medium, these cells have limited access to nutrients in the medium and have difficulty in exchanging waste into the medium. In addition, this reduced access to the medium makes cells in clusters unsuitable for genetic manipulation by factors introduced into the medium (i.e., for transformation by nucleic acids). As a result of these difficulties, 293 cells have not preferably been used in suspension culture for the production of biological materials. Even in 293 cells specially adapted for suspension culture a “lag” is observed from a high concentration passage to the next passage. For example, for particular cells if the cell density is allowed to overgrow above, for example, 2×10⁶ cells, several subsequent passages will be unable to achieve this density, but may only achieve half that density or less.

U.S. Pat. No. 4,767,704 (1988) to Cleveland recognized that trace elements were advantageously used in serum free media. Perhaps, this was because serum contained a sampling of trace metals whose absence was deleterious to mammalian culture. Cleveland noted difficulties of serum free culture:
Accordingly, previously investigators have attempted to grow hybridoma lines in media free of serum. However, other proteins and macromolecules were always added to these media in order to allow long-term growth of the hybridoma lines. For example, serum-free media supplemented with serum proteins such as transferrin and insulin (Chang et al, J. Immunol. Methods, 39: 369 (1980)) or transferrin and insulin plus albumin containing liposomes (Andersson and Melchers, Curr. Top. Microbiol. Immunol., 81: 130 (1978)) have been used. Another medium used the dialyzable fraction of serum for small-scale cultivation with a two-chambered Marbrook vessel (Klinman and McKeam, J. Immunol. Methods, 42: 1 (1981)). Serum proteins were avoided in still another medium, but cultivation was restricted to 24-48 hours because of cell death (Galfre and Milstein, Methods in Enzymology, Vol. 73B, eds. Colowick and Kaplan, Academic Press, New York, page 1, 1981). However, none of these methods allows for the routine large-scale production of monoclonal antibodies in a protein-free culture medium, and such a production process and culture medium are still needed.
   While Cleveland identified some benefit of trace elements at low concentrations for maintaining cells in serum free culture, the present invention recognizes that although at higher concentrations toxic effect might be observed, addition of higher total concentration of trace compounds can overcome observed toxicity and in some cases prolong culture time for example with copper and allow overcoming or avoiding a lag effect for example with zinc.

One important trace element, selenium, has long been recognized as an anti-oxidant by its participation in glutathione oxidative reactions. Several seleno proteins have also been investigated for their contributions to homeostasis in living organisms. Selenoprotein P has been reported to possess antioxidant activities and the ability to promote neuronal cell survival. Recent studies in cell culture and gene knockout models support a function for selenoprotein P in delivery of selenium to the brain. mRNAs for other selenoproteins, including selenoprotein W, thioredoxin reductases, 15-kDa selenoprotein and type 2 iodothyronine deiodinase, are also detected in the brain. Future research directions will surely unravel the important functions of this class of proteins in the brain. Chen J., Berry M J., J. Neurochem. 2003 July; 86(1):1-12. Although trace elements have been recognized as useful ingredients, especially in serum free and chemically defined culture, before the present invention serum free culture suffered from poor yields in comparison to serum containing culture. Also perplexing was the variability observed in different culture mixtures made to ostensibly be identical mixtures. The trace element mixes previously proposed have not met the needs of the cell culture arts, especially the serum free or chemically defined culture arts.

Thus, there still remains a need in the art for a cell medium that permits the growth of eukaryotic cells in suspension while permitting the transfection of the cells with a reduced amount of manipulation. Such a medium should preferably be a serum-free and/or chemically defined and/or protein-free medium and/or a medium lacking animal derived materials which facilitates the growth of mammalian cells to high density and/or increases the level of expression of recombinant protein, reduces cell clumping, and which does not require supplementation with animal proteins, such as serum, transferrin, insulin and the like. Preferably a medium of this type will permit the suspension cultivation of mammalian cells that are normally anchorage-dependent, including epithelial cells and fibroblast cells, such as 293 cells and CHO cells. Such culture media will facilitate studies of the effects of growth factors and other stimuli on cellular physiology, will allow easier and more cost-effective production and purification of biological substances (e.g., viruses, recombinant proteins, etc.) produced by cultured mammalian cells in the biotechnology industry, and will provide more consistent results in methods employing the cultivation of mammalian cells. These needs and others are met by the present invention.

### SUMMARY OF THE INVENTION

In the description that follows, a number of terms conventionally used in the field of cell culture media are utilized extensively. In order to provide a clear and consistent understanding of the specification and claims, and the scope to be given such terms, the following definitions are provided.

The term “ingredient” refers to any compound, whether of chemical or biological origin, that can be used in cell culture media to maintain or promote the growth of proliferation of cells. The terms “component,” “nutrient” and “ingredient” can be used interchangeably and are all meant to refer to such compounds. Typical ingredients that are used in cell culture media include amino acids, salts, metals, sugars, lipids, nucleic acids, hormones, vitamins, fatty acids, proteins and the like. Other ingredients that promote or maintain cultivation of cells ex vivo can be selected by those of skill in the art, in accordance with the particular need.

By “derivative” is meant a progeny of a cell, a descendant of a cell, a fusion product of a cell with another body, e.g., another cell, an organelle of a cell, e.g., a nucleus or other assemblage of biomolecules that retains characteristics of interest of a cell. A derivative of a chemical compound is a compound possessing the same function, but that is slightly altered, for example by ionization in solution, being formed as a salt, being formed as a crystal, being combined with another compound such as a hydrochloride, being hydroxylated or dehydroxylated, etc., or sometimes in the case of for example proteins, the function may be altered for example by cleaving a pro-form or the protein.

By “cell culture” or “culture” is meant the maintenance of cells in an artificial, in vitro environment. It is to be understood, however, that the term “cell culture” is a generic term and may be used to encompass the cultivation not only of individual cells, but also of tissues, organs, organ systems or whole organisms, for which the terms “tissue culture,” “organ culture,” “organ system culture” or “organotypic culture” may occasionally be used interchangeably with the term “cell culture.” By “cultivation” is meant the maintenance of cells in vitro under conditions favoring growth, differentiation or continued viability, in an active or quiescent state, of the cells. In this sense, “cultivation” may be used interchangeably with “cell culture” or any of its synonyms described above. Cells may be cultured attached or in suspension. The density of cells will refer to either the number of cells per given area or volume. For economic reasons higher densities are generally more desirable up until the point where cell growth or bioproduction is inhibited. A Culture density for larger cells is generally less than that for smaller cells such as bacteria. For example, mammalian cells are desirably cultured in suspension to a maximum density of about 10⁶, 2×10⁶, 2.5×10⁶, 3×10⁶, 3.5×10⁶, 4×10⁶, 4.5×10⁶, 5×10⁶, 6×10⁶, 7×10⁶, 10⁶, 9×10⁶, 10×10⁶, 11×10⁶, 12×10⁶, or preferably greater if proper conditions are achieved.

By “culture vessel” is meant a vessel, e.g., glass, plastic, or metal container that can provide an aseptic environment for culturing cells.

The phrases “cell culture medium,” “culture medium” (plural “media” in each case) and “medium formulation” refer to a nutritive solution for cultivating cells and may be used interchangeably. A cell in “culture” is a cell that is situated in a medium and environment intended to permit growth and/or maturation. While certain processes, such as centrifuging, filtration, etc., the cells, may be performed in the culture process the transient concentration preliminary to further dilution is not included in the cell density values considered herein.

The term “contacting” refers to the placing of cells to be cultivated in vitro into a culture vessel with the medium in which the cells are to be cultivated. The term “contacting” encompasses mixing cells with medium, pipetting medium onto cells in a culture vessel, and/or submerging cells in culture medium.

The term “combining” refers to the mixing or admixing of ingredients in a cell culture medium formulation.

A “trace element” is an element that is resent in only a trace concentration. A trace concentration may be less than a level ordinarily or easily measured, for example the trace level may be <10⁻⁵, <10⁻⁶, <10⁻⁷ or <10⁻⁸M. The trace elements of the present invention are preferably present as ions or chelated complexes. The ions may be simple ions comprising only a single element or may be complex ions comprising two or more elements. Preferably the elements are transition metal elements, e.g., elements selected from the group consisting of Sc, Ti, V, Cr, Mn, Fe, Co, Ni, Cu, Zn, Ga, As, Se, Br, Al, Si, P, Y, Zr, Nb, Mo, Tc, Ru, Rh, Rb, Ce, Ag, Pd, Ag, Cd, In, Sn, Sb, F, Te, Au, Pt, Bi, Ir, Os, Re, W, Ta and Hf. Some elements may be present at more than trace amounts, i.e., >10⁻⁵ in a 1× concentration, in which case that element, e.g., Fe or Zn would not be considered a trace element, but may nonetheless be advantageously used with or as part of the present invention and thus may be included specifically in some aspects of the invention.

Except where obvious otherwise from the context “serum free” is used as a shorthand for various culture conditions defined as serum free, protein free, animal origin free and/or chemically defined. “Chemically defined” is a preferred class of media within the category of “serum free” as used herein.

“Trace metals”, “trace elements” and “trace compounds” are used interchangeably. While in many cases, the trace element of interest will be present as a complex ion, the precise species of the one or more species of ions resulting form addition of salt to the medium solvent is not of interest for the present invention. Since many of the trace minerals are also transition metals occasionally, “metals” or “transition metals” will be an alternate term having the same meaning. Each of these terms includes the reaction products resulting form their use in the medium.

As used herein “lag” is a phenomenon sometimes observed when a cell density in a culture is permitted to exceed a threshold density. When exceeding the threshold density prevents one or more subsequent passages from meeting or exceeding the threshold density or a density that is a fraction of said threshold density, “lag” is said to be occurring.

As used herein, a “trace” element will be said to be present when it is intentionally present, either by known addition to the medium or intentional use of a nonpure material for the purpose of adding the trace “impurity”. Thus when a medium “comprises” for example, Cd, the medium will have a medium known quantity of Cd (because a known amount was added either as a known avoidable impurity or as a component) but may contain more Cd (because of unknown impurities), or slightly less Cd (because small quantities may have been sequestered by containers, utensils etc.). Cd may be a free ion in culture, either intracellular or extracellular, or may be complexed, for example, by binding to a protein or other biomolecule or complex. Thus a medium will be said not to comprise, e.g., Cd, even though miniscule quantities might be suspected or unintentionally introduced.

A cell culture medium is composed of a number of ingredients and these ingredients vary from one culture medium to another. Generally a cell culture medium will have solutes dissolved in solvent. The solutes provide an osmotic force to balance the osmotic pressure across the cell membrane (or wall). Additionally the solutes will provide nutrients for the cell. Some nutrients will be chemical fuel for cellular operations; some nutrients may be raw materials for the cell to use in anabolism; some nutrients may be machinery, such as enzymes or carriers that facilitate cellular metabolism; some nutrients may be binding agents that bind and buffer ingredients for cell use or that bind or sequester deleterious cell products. Depending on the cell and the intended use of the cell, these ingredients will optimally be present at concentrations balanced to optimize cell culture performance. Performance will be measured in accordance with a one or more desired characteristics, for example, cell number, cell mass, cell density, O₂ consumption, consumption of a culture ingredient, such as glucose or a nucleotide, production of a biomolecule, secretion of a biomolecule, formation of a waste product or by product, e.g., a metabolite, activity on an indicator or signal molecule, etc. Each or a selection of the ingredients will thus preferably optimized to a working concentration for the intended purpose.

A “1× formulation” is meant to refer to any aqueous solution that contains some or all ingredients found in a cell culture medium at working concentrations. The “1× formulation” can refer to, for example, the cell culture medium or to any subgroup of ingredients for that medium. The concentration of an ingredient in a 1× solution is about the same as the concentration of that ingredient found in a cell culture formulation used for maintaining or cultivating cells in vitro. A cell culture medium used for the in vitro cultivation of cells is a 1× formulation by definition. When a number of ingredients are present, each ingredient in a 1× formulation has a concentration about equal to the concentration of those ingredients in a cell culture medium. For example, RPMI-1640 culture medium contains, among other ingredients, 0.2 g/L L-arginine, 0.05 g/L L-asparagine, and 0.02 g/L L-aspartic acid. A “1× formulation” of these amino acids contains about the same concentrations of these ingredients in solution. Thus, when referring to a “1× formulation,” it is intended that each ingredient in solution has the same or about the same concentration as that found in the cell culture medium being described. The concentrations of ingredients in a 1× formulation of cell culture medium are well known to those of ordinary skill in the art. See Methods For Preparation of Media, Supplements and Substrate For Serum-Free Animal Cell Culture Allen R. Liss, N.Y. (1984), which is incorporated by reference herein in its entirety. The osmolarity and/or pH, however, may differ in a 1× formulation compared to the culture medium, particularly when fewer ingredients are contained in the 1× formulation.

A “10× formulation” is meant to refer to a solution wherein each ingredient in that solution is about 10 times more concentrated than the same ingredient in the cell culture medium. For example, a 10× formulation of RPMI-1640 culture medium may contain, among other ingredients, 2.0 g/L L-arginine, 0.5 g/L L-asparagine, and 0.2 g/L L-aspartic acid (compare 1× formulation, above). A “10× formulation” may contain a number of additional ingredients at a concentration about 10 times that found in the 1× culture medium. As will be readily apparent, “25× formulation,” “50× formulation,” “100× formulation,” “500× formulation,” and “1000× formulation” designate solutions that contain ingredients at about 25-, 50-, 100-, 500-, or 1000-fold concentrations, respectively, as compared to a 1× cell culture medium. Again, the osmolarity and pH of the media formulation and concentrated solution may vary. A formulation may contain components or ingredients at 1× with respect to a particular cell culture protocol, but at a concentration, for example, 2, 2.5, 5, 6.7, 9, 12 etc. x with respect to a different culture protocol or different base medium. A formulation may be a complete formulation, i.e., a formulation that requires no supplementation to culture cells, may be an incomplete formulation, i.e., a formulation that requires supplementation or may be a supplement that may supplement an incomplete formulation or in the case of a complete formulation, may improve culture or culture results.

The present invention may be used in any culture process, but is especially preferred for eukaryotic cells, especially biomolecule producing cells, microorganisms, such as yeast, e.g., filametous yeasts, insect cells, fish cells, avian cells and mammalian cells. Bioproduction may include vaccine production, protein production, glycoprotein production, liprotein production, antibody or antigen production, nucleic acid production, organelle production, lipid production, carbohydrate production, etc. Preferably the biomolecule produced will be produced in a less expensive, more efficient or other advantageous manner. Mammalian cells, including primary epithelial cells (e.g., keratinocytes, cervical epithelial cells, bronchial epithelial cells, tracheal epithelial cells, kidney epithelial cells and retinal epithelial cells) and established cell lines and their strains (e.g., 293 embryonic kidney cells, A-549, Jurkat, Namalwa, Hela, 293BHK cells, HeLa cervical epithelial cells and PER-C6 retinal cells, aka PER.C6, MDBK (NBL-1) cells, 911 cells, CRFK cells, MDCK cells, CHO cells, BeWo cells, Chang cells, Detroit 562 cells, HeLa 229 cells, HeLa S3 cells, Hep-2 cells, KB cells, LS 180 cells, LS 174T cells, NCI-H-548 cells, RPMI 2650 cells, SW-13 cells, T24 cells, WI-28 VA13, 2RA cells, WISH cells, BS-C-I cells, LLC-MK₂ cells, Clone M-3 cells, 1-10 cells, RAG cells, TCMK-1 cells, Y-1 cells, LLC-PK₁ cells, PK(15) cells, GH₁ cells, GH₃ cells, L2 cells, LLC-RC 256 cells, MH₁C₁ cells, XC cells, MDOK cells, VSW cells, and TH-I, B1 cells, or derivatives thereof), fibroblast cells from any tissue or organ (including but not limited to heart, liver, kidney, colon, intestines, esophagus, stomach, neural tissue (brain, spinal cord), lung, vascular tissue (artery, vein, capillary), lymphoid tissue (lymph gland, adenoid, tonsil, bone marrow, and blood), spleen, and fibroblast and fibroblast-like cell lines (e.g., CHO cells, TRG-2 cells, IMR-33 cells, Don cells, GHK-21 cells, citrullinemia cells, Dempsey cells, Detroit 551 cells, Detroit 510 cells, Detroit 525 cells, Detroit 529 cells, Detroit 532 cells, Detroit 539 cells, Detroit 548 cells, Detroit 573 cells, HEL 299 cells, IMR-90 cells, MRC-5 cells, WI-38 cells, WI-26 cells, MiCl₁ cells, CHO cells, CV-1 cells, COS-1 cells, COS-3 cells, COS-7 cells, Vero cells, DBS-FrhL-2 cells, BALB/3T3 cells, F9 cells, SV-T2 cells, M-MSV-BALB/3T3 cells, K-BALB cells, BLO-11 cells, NOR-10 cells, C₃H/IOTI/2 cells, HSDM₁C₃ cells, KLN205 cells, McCoy cells, Mouse L cells, Strain 2071 (Mouse L) cells, L-M strain (Mouse L) cells, L-MTK (Mouse L) cells, NCTC clones 2472 and 2555, SCC-PSA1 cells, Swiss/3T3 cells, Indian muntjac cells, SIRC cells, C_{II} cells, and Jensen cells, or derivatives thereof) all may benefit in culture from the present media. More preferably, the medium is used to culture mammalian cells selected from the group consisting of 293 cells, PER-C6 cells, CHO hells, COS cells and Sp2/0 cells. More preferably, the medium is used to culture 293 cells, Mimic cells or Per.C6 cells. Preferably, the medium is used to culture cells in suspension.

While profound effects of the present invention have been observed in eukaryotic cells, the present invention is also applicable to bacterial cells. Bacterial cultures tend to be tolerant of non-optimal conditions, such as impurities, temperature, osmolarity, light, solvents, nutrients present, etc. However, even though bacterial cultures may be functional in non-optimal conditions, improvements are available by practicing the present invention using microbial, e.g., bacterial cultures.

In one aspect, the present invention provides a serum-free, eukaryotic cell culture medium supplement comprising or obtained by combining one or more ingredients selected from the group consisting of one or more antioxidants, one or more albumins or albumin substitutes, one or more lipid agents, one or more insulins or insulin substitutes, one or more transferrins or transferrin substitutes, one or more trace elements, and one or more glucocorticoids, wherein a basal cell culture medium supplemented with the supplement is capable of supporting the expansion of cells, for example, CD34⁺ hematopoietic cells and cells of myeloid lineage, 293 embryonic kidney cells, A-549, Jurkat, Namalwa, Hela, 293BHK cells, HeLa cervical epithelial cells and PER-C6 retinal cells, aka PER.C6, MDBK (NBL-1) cells, 911 cells, CRFK cells, MDCK cells, CHO cells, BeWo cells, Chang cells, Detroit 562 cells, HeLa 229 cells, HeLa S3 cells, Hep-2 cells, KB cells, LS 180 cells, LS 174T cells, NCI-H-548 cells, RPMI 2650 cells, SW-13 cells, T24 cells, WI-28 VA13, 2RA cells, WISH cells, BS-C-I cells, LLC-MK₂ cells, Clone M-3 cells, 1-10 cells, RAG cells, TCMK-1 cells, Y-1 cells, LLC-PK₁ cells, PK(15) cells, GH₁ cells, GH₃ cells, L2 cells, LLC-RC 256 cells, MH₁C₁ cells, XC cells, MDOK cells, VSW cells, and TH-I, B1 cells, or derivatives thereof), fibroblast cells from any tissue or organ (including but not limited to heart, liver, kidney, colon, intestines, esophagus, stomach, neural tissue (brain, spinal cord), lung, vascular tissue (artery, vein, capillary), lymphoid tissue (lymph gland, adenoid, tonsil, bone marrow, and blood), spleen, and fibroblast and fibroblast-like cell lines (e.g., CHO cells, TRG-2 cells, IMR-33 cells, Don cells, GHK-21 cells, citrullinemia cells, Dempsey cells, Detroit 551 cells, Detroit 510 cells, Detroit 525 cells, Detroit 529 cells, Detroit 532 cells, Detroit 539 cells, Detroit 548 cells, Detroit 573 cells, HEL 299 cells, IMR-90 cells, MRC-5 cells, WI-38 cells, WI-26 cells, MiCl₁ cells, CHO cells, CV-1 cells, COS-1 cells, COS-3 cells, COS-7 cells, Vero cells, DBS-FrhL-2 cells, BALB/3T3 cells, F9 cells, SV-T2 cells, M-MSV-BALB/3T3 cells, K-BALB cells, BLO-11 cells, NOR-10 cells, C₃H/IOTI/2 cells, HSDM₁C₃ cells, KLN205 cells, McCoy cells, Mouse L cells, Strain 2071 (Mouse L) cells, L-M strain (Mouse L) cells, L-MTK (Mouse L) cells, NCTC clones 2472 and 2555, SCC-PSA1 cells, Swiss/3T3 cells, Indian muntjac cells, SIRC cells, C_{II} cells, and Jensen cells, or derivatives thereof). More preferably, the medium is used to culture cells selected from the group consisting of 293 cells, PER-C6 cells, CHO cells, COS cells and Sp2/0 cells, Mimic cells or Per.C6 cells and/or derivatives thereof in serum-free culture.

The present invention also provides a serum-free, eukaryotic cell culture medium supplement comprising or obtained by combining one or more antioxidants and one or more ingredients selected from the group consisting of one or more albumins or albumin substitutes, one or more growth factors, one or more lipid agents, one or more insulins or insulin substitutes, one or more transferrins or transferrin substitutes, one or more trace elements, and/or one or more glucocorticoids, wherein a basal cell culture medium supplemented with the supplement is capable of supporting the expansion of cells in serum-free culture.

The present invention also specifically provides a serum-free, eukaryotic cell culture medium supplement comprising or obtained by combining one or more ingredients selected from the group consisting of N-acetyl-L cysteine, human serum albumin, Human Ex-Cyte.R™., ethanolamine HCl, human zinc insulin, human iron saturated transferrin, Se⁴⁺, hydrocortisone, D,L-tocopherol acetate, and 2-mercaptoethanol, and a trace element mix, wherein the ingredients are present in an amount which, when the supplement is added to a basal cell culture medium, supports the expansion of cells in serum-free culture.

The present invention also provides a method of making a serum-free, eukaryotic cell culture medium supplement, the method comprising admixing water, N-acetyl-L cysteine, human serum albumin, Human Ex-Cyte™, ethanolamine HCl, human zinc insulin, human iron saturated transferrin, trace elements (e.g., a Se⁴⁺ salt), hydrocortisone, D,L-tocopherol acetate, and/or 2-mercaptoethanol and a trace element mix, wherein each ingredient is present in an amount which, when added to a basal medium, supports cells in serum-free culture.

The present invention provides medium or a supplement that when added to basal medium improves cell culture. Improved culture may be exhibited by more rapid cell growth, decreased doubling time, higher achievable density of cells, higher production or yield of biomolecule, such as protein, e.g., antibody or other proteins of therapeutic interest.

The present invention provides a method for producing amounts of biomolecules of interest at a concentration exceeding 4, 5, 6, 7, 8, 9, 10 11, 12 14, 15, 18 or 20 mg/ml. Depending on the molecule and cell type different cell densities are achievable and different yields are achievable. A reasonable estimate of the amount of biomolecule in the culture can be obtained by multiplying the cell density by between 0.05 and 0.2 mg/10⁶ vc. Other investigators have modified or engineered cells to produce higher titers of biomolecule and in some cells to achieve higher cell densities that could further benefit from the present invention. Thus biomolecule concentrations at high cell counts in high producing cells may be found at 10 to 100 mg/ml.

For microbial, e.g., fungal, low eukaryotic and bacterial, cell culture that also can benefit form the compositions and methods cells are often counted by other means such as by light scattering. Much higher densities for example 10 fold or 100 fold and sometimes up to 1000 fold higher of biomolecules can be obtained. These cultures can still benefit from practicing the instant invention.

The present invention also provides a kit comprising a carrier means, the carrier means being compartmentalized to receive in close confinement therein one or more container means, wherein a first container means contains a supplement of the present invention, and wherein optionally a second container means contains a basal medium. The carrier means may or may not be stored or shipped as a single kit, but the kit may be in separate containers not contained by a larger container. For example, the kit may include a large reservoir for a basal medium and a trace element concentrate to be added as a supplement to the medium. The medium may be provided as a 1× liquid form, may be a concentrate, may be a dry powder, including dry format powder such as AGT powder. The present invention also provides a eukaryotic cell culture medium obtained by combining a basal cell culture medium with a supplement of the invention, wherein the medium is capable of supporting cells in culture, preferably, serum-free culture.

The present invention also provides a culture including a cell culture medium containing one or more cells, e.g., a mammalian cell. The cell culture medium of the invention is not limited to any particular cell type, but may be put to advantageous use in culturing any cell with special requirements met by the present invention. A composition of the present invention may be made by adding trace compounds other than one of Ag or Ni in at least a five fold excess to the Ag or the Ni or may be made by adding trace compounds other than one of Co, Mn, Ag or Ni in at least a five fold excess to the Co, Mn, Ag or the Ni. The present invention also provides embodiments of compositions for use in growing cells, said compositions comprising trace compounds wherein a sum of concentrations of said trace compounds not including Zn is less than or less than about 3×10⁻⁷M or less than or less than about 6×10⁻⁷M including Zn or a sum not including Fe and/or Zn is less than or less than about 3×10⁻⁷M or less than or less than about 6×10⁻⁷M including Zn. Another embodiment of the present invention provides a method of growing cells in culture comprising providing a trace compound composition of the present invention to a cell in a culture container and culturing said cell. Yet another embodiment of the present invention provides a method for inhibiting apoptosis of cells grown in culture comprising culturing cells in a composition of the present invention or in a presence of a medium according to the present invention.

The present invention also provides a culture medium or medium supplements containing one or more, preferably 2, 3, 4, 5, 6, 7, 8, 9, 10, 11, 12, 13, 14, 15, 16, 17, 18, 19, 20, 21, or 22 elements selected from the group consisting of copper, iron, zinc, manganese, silicon, molybdenum, vanadium, nickel, tin, aluminum, silver, barium, bromine, cadmium, cobalt, chromium, fluorine, germanium, iodine, rubidium, zirconium, or selenium. Especially preferred are the elements Cu, Zn, and Se. For example, a preferred supplement of the present invention contains copper, zinc, vanadium, germanium molybdenum, manganese, selenium, zirconium and optionally one or more of rubidium, cadmium, aluminum, cobalt, nickel, barium, silver or titanium. Especially preferred media of the present invention contain copper, zinc, and selenium, and optionally zirconium, barium, titanium and/or germanium.

The present invention also provides a serum-free eukaryotic cell culture medium comprising one or more ingredients selected from the group consisting of one or more antioxidants, one or more inorganic salts, one or more energy sources, one or more buffering agents, one or more amino acids, additionally one or more trace element, and optionally one or more albumins or albumin substitutes, one or more lipid agents one or more insulins or insulin substitutes, one or more transferrins or transferrin substitutes, one or more trace elements, one or more glucocorticoids, one or more pyruvate salts, one or more pH indicators, one or more vitamins, wherein the medium is capable of supporting the expansion cells in culture, preferably in serum-free culture.

### BRIEF DESCRIPTION OF THE DRAWINGS

FIG. 01 shows the toxic effects on PER.C6 cell growth in AEM to which cobalt, nickel and manganese have been added. The extension of the growth phase resulting from addition of copper is also demonstrated. Cobalt, Nickel and Manganese at low concentration are shown to be all individually toxic to Per.C6™ cells cultured in AEM. Copper at a low concentration is shown to increase growth of Per.C6™ cells cultured in AEM.

FIG. 02 shows the elimination of batch-to-batch variability in multiple batches of AEM by the addition of TEM-2.

FIG. 03 shows the consistent performance in a batch of AEM to which TEM-2 has been added at 0.5×, 1× and 2× concentrations.

FIG. 04 shows the elimination of the toxicity of cobalt, nickel and manganese through supplementation with TEM-2.

FIG. 05 shows the increased performance of AEM supplemented with TEM-3 as compared to TEM-2. It also demonstrates the equivalent performance of two batches of AEM produced with TEM-3 added at the time of production.

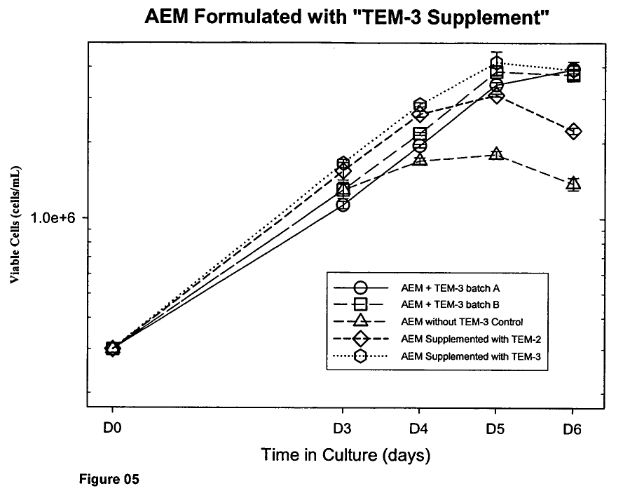

FIG. 06 shows the elimination of the post high-density lag through supplementation of the TEM-3. The addition of TEM-3 to 293 SFM II eliminated the “Lag” demonstrated when sub culturing from a high-density culture.

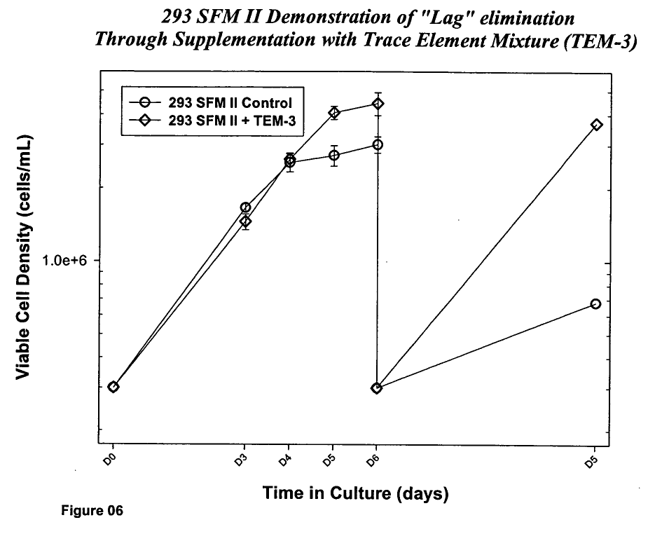

FIG. 07 shows high antibody production and cell density demonstrated by preferred embodiments of the present invention.

### DETAILED DESCRIPTION OF THE INVENTION

The present invention concerns trace minerals or trace elements. Trace elements are elements that are present in negligible, sometimes undetectable or unquantifiable amounts. Cells in culture require an osmotic pressure that balances the intracellular forces with the exterior. The bulk of the outside ingredients has traditionally been monovalent cations and anions and amino acids. Often sodium chloride is the major ingredient. Other common ingredients include lipids, vitamins, antioxidants chelators, divalent cations, buffers and sugars. Plant or animal hydrolysates are also sometimes used. One shortcoming of eukaryotic cell culture is a lack of consistency that is observed when different batches (manufacturing runs) of ostensibly the same medium formulation are used to culture cells. This lack of consistency has been especially problematic in attempted production of therapeutic biomolecules because of the large scale, but also plagues scientific research where repeatability is a requirement for science to be accepted by the community. Culture using insect cells and mammalian cells are most noted for the variability probably because these cell types are preferred for bioproduction of complex biomolecules, but other cells, such as yeast cells, for example filamentous yeast, also could see culture improvement by increasing batch to batch or lot to lot consistency. As more biomolecules are used for therapeutics and diagnostics and for industrial purposes, such as enzyme production, variability will be a problem of increasing concern.

Trace metals have been used in previous cultures. For example the following oxidation states have been preferred: Cu²⁺, Fe²⁺ and Fe³⁺, Zn²⁺, Mn²⁺, SiO₃²⁻, MoO₄²⁻, VO₄³⁻, Ni²⁺, Sn²⁺, Al³⁺, Ag⁺, Ba²⁺, Br—, Cd²⁺, Co²⁺, Cr⁶⁺, F⁻, Ge⁴⁻, I⁻, Rb⁺, Zr⁴⁺, and SeO₃²⁻. Other compounds including other compounds with different oxidation states since the cell machinery can both oxidize and reduce, can be used if, for example, cost, availability, solubility or other concerns calls for the other compound. Since these elements are present is low concentrations, sometimes below easily detectable amounts and because the concentrations of each are subject to variability during manufacture, e.g., from leaching or sequestration by equipment (containers, tubing, filters, variability of concentrations in even ultra pure raw materials, purified water supplies, etc.) these elements are suspect as a possible cause of variability between different manufacturing batches. Since the trace compounds are often not added intentionally to media sometimes below concentrations easily detectable or economically or confidently removed, the actual concentration is often not known. It is expected that improved or rigorous analytical means would detect an individual trace compound or several trace compounds in media ostensibly free of such. Beneficial trace elements range from metals to non-metals. The variable oxidation state appears to be an important factor is their contributions positive and negative to cell culture. Many trace elements are important participants with or cofactors of oxidative-reduction enzymes in the body. Many also have roles in transport proteins, cofactors, and detoxification and immunological and chemical defense. For example, selenium is a cofactor of glutathione an important oxidant scavenger in the human body. Zn has been recognized as an essential element for immune function though the precise mechanisms of action are not known. Significant contributions of trace elements are carried bound to transport proteins in the blood. Hence serum often contributes a sufficient or an overwhelming concentration of trace elements. Many trace elements are toxic when in free form. The following general discussion of trace elements is meant as context and is not considered a discovery by the present inventor.

Iron

Iron is important in the transportation of oxygen in red blood cells by way of the blood stream to the tissues. Iron is present in the protein, hemoglobin. A similar protein in muscle, myoglobin, also contains iron and stores oxygen for use during muscle contraction. Iron is found in the portion of the cell involved in energy production and as a cofactor for several enzymes. Iron is active in lipid peroxidation observed in the liver and other organs.

Zinc

Zinc is important for proper functioning of the immune system. Zinc is a cofactor for many enzymes, which means that zinc is necessary for the proper functioning of these enzymes. These enzymes participate in the metabolism of carbohydrates, lipids, proteins and nucleic acids (such as DNA). Zinc is involved in functioning of the immune system and in the expression of genetic information. Zinc is also present in members of a class of proteins called the metallothioneins that are believed to provide antioxidant protection by scavenging free radicals. Excessive zinc interferes with the function of copper and iron.

Iodine

Iodine is present in the thyroid gland which acts as a reservoir within the organism. Iodine is believed to participate in some secretory pathways.

Chromium

Chromium is essential for carbohydrate, fat, and nucleic acid (DNA or RNA) metabolism. Chromium is part of the glucose tolerance factor (GTF) that is required for insulin action. Chromium also appears to affect some of the enzymes that regulate cholesterol synthesis, one of the effects on lipid metabolism. Although probably related to the effects on lipids and thus the lipid membranes of cells such as nerve cells, the mechanism by which chromium participates in proper nerve function is not well understood.

Cobalt

Cobalt has a central action in vitamin B₁₂ function. It is not known if cobalt has other functions. An RDA has not been established. At large concentrations it also interferes with the activity of iron.

Copper

Copper is incorporated into many enzymes and is necessary for their actions. For example, the copper containing ceruloplasmin is involved in the transport of iron in the blood to places where hemoglobin synthesis occurs.

Manganese

Manganese assists in the activity of many enzymes, including some involved in lipid, protein, and carbohydrate metabolism.

Molybdenum

Molybdenum is part of the molecular structure of several enzymes. One of these enzymes is involved in the formation of sulfate. An excess of molybdenum interferes with copper and iron absorption, but interactions in media are not well documented.

Selenium

Selenium is an essential nonmetallic element. Selenium is important for the function of several proteins. One of these is glutathione peroxidase, an enzyme that prevents oxidative damage to cells from a variety of peroxides. Selenium also appears to bind to some minerals such as arsenic and mercury and decrease their toxicity.

Nickel

Although nickel is an essential element for animal nutrition, the physiologic role of nickel is not yet established.

General speculation relates to some or the other trace compounds. Some purported attributes attributed to several above and to others not listed above are listed below. However, the attributions relating to intact organisms may not be directly relevant to the function in in vitro culture. Although function may not be known the trace compounds and/or combinations thereof may still be advantageously used in a serum free, even a chemically defined medium.

Calcium: Supports the maintenance of healthy blood pressure levels; Supports the maintenance of healthy bone mass.

Magnesium: Supports the maintenance of a healthy heart; Supports the maintenance of healthy blood pressure levels.

Zinc: Supports cell respiration; Supports DNA and RNA replication; Supports the functions of antioxidants; Supports the immune system.

Selenium: Supports the maintenance of normal cell functions; Supports cell respiration; Supports DNA and RNA replication; Supports the functions of antioxidants.

Copper: Supports the health of the heart; Supports the maintenance of healthy cell respiration; Supports DNA and RNA replication; Supports the functioning of antioxidants.

Manganese: Supports the maintenance of healthy bone mass; Supports the maintenance of a healthy reproductive system.

Chromium: Essential trace element; Supports the body's efforts to maintain normal glucose levels.

Molybdenum: Supports cellular respiration; Supports DNA and RNA replication; Supports the functioning of antioxidants.

Barium: Barium inhibits the endothelium-dependent component of flow but not acetylcholine-induced relaxation in isolated rabbit cerebral arteries.

Boron: Supports the maintenance of healthy bone mass.

Gadolinium: Supports healthy cellular functions.

Antimony: Supports the health of the body.

Neodymium: Supports the maintenance of healthy circulation; Supports the maintenance of normal cellular functions.

Lutetium: Modulates DNA metabolism.

Holmium: Supports normal cellular functions.

Thalium: Supports healthy cellular functions.

Terbium: Supports normal cellular functions.

Scandium: Supports normal cellular functions.

Erbium: Supports normal cellular functions.

Zirconium: Supports the health of the body; Low toxicity.

Ytterbium: Supports normal cellular functions.

Tellurium: Trace mineral.

Rubidium: Competes with Potassium ions in ion channels; Supports the health of the body.

Hafnium: Supports the health of the body.

Yttrium: Supports the maintenance of normal cellular functions; Supports the maintenance of youthful feelings.

Cerium: Supports the assimilation of amino acids.

Samarium: Supports the maintenance of healthy cellular functions.

Sulfur: Supports the maintenance of healthy cells; Supports collagen formation.

Praseodymium: Supports normal cellular functions.

Cesium: Competes with Potassium ions in ion channels.

Silver: Vital antibacterial, anti-infective, a natural antibiotic.

Lanthanum: Lanthanum inhibits steady-state turnover of the sarcoplasmic reticulum calcium ATPase by replacing magnesium as the catalytic ion.

Germanium: Antioxidant.

Europium: Supports healthy cellular functions.

Dysprosium: Supports normal cellular functions.

Rhodium: Supports the maintenance of normal cell functions.

Rhenium: Steric crowding around rhenium inhibits reactions of larger dienophiles.

Titanium: Supports the health of the body.

Ruthenium: Trace mineral.

Palladium: Supports the health of the body.

Niobium: Trace mineral.

Iridium: Supports the maintenance of normal cell functions.

Bismuth: Supports the digestive tract.

Tungsten: Trace mineral.

Thallium: Thalium binds to ferritin, but not apo-ferritin.

Tantalum: Trace mineral.

Strontium: Ionic strontium forms colloidal or particulate strontium phosphate, or binds to plasma proteins to form partly diffusible complexes.

Platinum: Supports cellular functions.

Gold: Supports the body against minor inflammation.

Beryllium: Trace mineral.

Tin: Supports the immune system; Supports the health of the body.

Thorium: Supports the assimilation of amino acids.

Indium: Indium pretreatment of rats and mice has been reported to decrease the concentration of cytochrome P-450, thereby reducing the activity of some cytochrome P-450 dependent enzymatic reactions.

Gallium: Supports the maintenance of cellular health.

Vanadium: Supports the maintenance of healthy blood sugar/insulin levels; Supports the body's efforts to lower cholesterol; Supports the maintenance of normal cell functions; Vanadate has insulin-like effects in adipocytes without stimulating insulin receptor kinase activity; Powerful inhibitor of many, but not all enzymes that cleave the terminal phosphate bond of ATP.

Nickel: Supports the metabolism of folate.

Lithium: Supports the health of the nervous system.

Cobalt: Supports the functioning serotonin.

Bromide/Bromonium: Supports the nervous system.

The trace compounds are preferably a mixture of trace compounds. One or more of the above or other trace elements may be advantageously used in cell culture. Addition of trace elements appears to mitigate or overcome toxic or deleterious effects of trace elements already present. Since it is virtually impossible to eliminate all trace elements, it cannot be said precisely what toxic levels are or to attribute a specific toxic pathway to a given concentration of trace element. As set forth in the examples, addition of a small amount of trace material may actually inhibit cells in culture, but a larger concentration may overcome the effect.

Many trace elements are known to have beneficial effect in cell culture. See e.g., Cleveland. The trace compounds shown in the table were selected for use in the present examples. The trace metals not listed in the table below are expected as a mixture to offer similar effect.

TABLE 1

| | | | measured |
|---|---|---|---|
| | working range (mol) | | concentration |
| COMPONENTS | min | max | mol |
| Aluminum Chloride 6H₂O | 2.25E−10 | 2.24896E−08 | 2.24896E−09 |
| Cadmium Chloride 2.5H₂O | 9.05E−09 | 3.61895E−07 | 9.04737E−08 |
| Rubidium Chloride | 5.24E−10 | 5.23967E−08 | 5.23967E−09 |
| Zirconium Chloride 8H₂O | 6.21E−10 | 6.21459E−08 | 6.21459E−09 |
| Cobalt Chloride 6H₂O | 1.82E−09 | 1.82437E−07 | 1.82437E−08 |
| Stannous Chloride 2H₂O | 4.49E−11 | 4.48673E−09 | 4.48673E−10 |
| Chromium Sulfate 15H₂O | 7.65E−11 | 7.65306E−09 | 7.65306E−10 |
| Nickelous Sulfate 6H₂O | 4.54E−11 | 4.53992E−09 | 4.53992E−10 |
| Sodium Flouride | 4.31E−09 | 4.30952E−07 | 4.30952E−08 |
| Cupric Sulfate 5H₂O | 1.81E−09 | 1.8096E−07 | 1.8096E−08 |
| Manganese Sulfate H₂O | 8.99E−11 | 8.99408E−09 | 8.99408E−10 |
| Ammonium Molybdate | 4.39E−10 | 4.39159E−08 | 4.39159E−09 |
| Germanium Dioxide | 2.30E−10 | 2.30476E−08 | 2.30476E−09 |
| Sodium Meta Vanadate | 4.59E−10 | 4.59016E−08 | 4.59016E−09 |
| Potassium Bromide | 4.55E−11 | 4.55462E−09 | 4.55462E−10 |
| Potassium Iodide | 5.01E−11 | 5.01205E−09 | 5.01205E−10 |
| Barium Acetate | 4.61E−10 | 4.61176E−08 | 4.61176E−09 |
| Silver Nitrate | 4.68E−11 | 4.68235E−09 | 4.68235E−10 |
| Titanium Tetrachloride | 1.37E−10 | 1.36842E−08 | 1.36842E−09 |
| Sodium Selenite | 9.08E−09 | 9.07861E−07 | 9.07861E−08 |
| Cupric Chloride 2H20 | 2.06E−08 | 2.05882E−06 | 2.05882E−07 |
| Zinc Chloride | 1.84E−07 | 1.83824E−05 | 1.83824E−06 |

TABLE 2

| | preferred range | | more preferred range | |
|---|---|---|---|---|
| COMPO- | | | | |
| NENTS | max mol | mol | min mol | max mol |
| Aluminum | 5.62E−10 | 8.99585E−09 | 1.12448E−09 | 4.49793E−09 |
| Chloride | | | | |
| 6H₂O | | | | |
| Cadmium | 2.26E−08 | 3.61895E−07 | 4.52368E−08 | 1.80947E−07 |
| Chloride | | | | |
| 2.5H₂O | | | | |
| Rubidium | 1.31E−09 | 2.09587E−08 | 2.61983E−09 | 1.04793E−08 |
| Chloride | | | | |
| Zirconium | 1.55E−09 | 2.48584E−08 | 3.1073E−09 | 1.24292E−08 |
| Chloride | | | | |
| 8H₂O | | | | |
| Cobalt | 4.56E−09 | 7.29748E−08 | 9.12185E−09 | 3.64874E−08 |
| Chloride | | | | |
| 6H₂O | | | | |
| Stannous | 1.12E−10 | 1.79469E−09 | 2.24336E−10 | 8.97345E−10 |
| Chloride | | | | |
| 2H₂O | | | | |
| Chromium | 1.91E−10 | 3.06122E−09 | 3.82653E−10 | 1.53061E−09 |
| Sulfate | | | | |
| 15H₂O | | | | |
| Nickelous | 1.13E−10 | 1.81597E−09 | 2.26996E−10 | 9.07985E−10 |
| Sulfate | | | | |
| 6H₂O | | | | |
| Sodium | 1.08E−08 | 1.72381E−07 | 2.15476E−08 | 8.61905E−08 |
| Flouride | | | | |
| Cupric | 4.52E−09 | 7.2384E−08 | 9.048E−09 | 3.6192E−08 |
| Sulfate | | | | |
| 5H₂O | | | | |
| Man- | 2.25E−10 | 3.59763E−09 | 4.49704E−10 | 1.79882E−09 |
| ganese | | | | |
| Sulfate | | | | |
| H₂O | | | | |
| Am- | 1.10E−09 | 1.75663E−08 | 2.19579E−09 | 8.78317E−09 |
| monium | | | | |
| Molybdate | | | | |
| Ger- | 5.76E−10 | 9.21905E−09 | 1.15238E−09 | 4.60952E−09 |
| manium | | | | |
| Dioxide | | | | |
| Sodium | 1.15E−09 | 1.83607E−08 | 2.29508E−09 | 9.18033E−09 |
| Meta | | | | |
| Vanadate | | | | |
| Potassium | 1.14E−10 | 1.82185E−09 | 2.27731E−10 | 9.10924E−10 |
| Bromide | | | | |
| Potassium | 1.25E−10 | 2.00482E−09 | 2.50602E−10 | 1.00241E−09 |
| Iodide | | | | |
| Barium | 1.15E−09 | 1.84471E−08 | 2.30588E−09 | 9.22353E−09 |
| Acetate | | | | |
| Silver | 1.17E−10 | 1.87294E−09 | 2.34118E−10 | 9.36471E−10 |
| Nitrate | | | | |
| Titanium | 3.42E−10 | 5.47368E−09 | 6.84211E−10 | 2.73684E−09 |
| Tetra- | | | | |
| chloride | | | | |
| Sodium | 2.27E−08 | 3.63145E−07 | 4.53931E−08 | 1.81572E−07 |
| Selenite | | | | |
| Cupric | 5.15E−08 | 8.23529E−07 | 1.02941E−07 | 4.11765E−07 |
| Chloride | | | | |
| 2H20 | | | | |
| Zinc | 4.60E−07 | 7.35294E−06 | 9.19118E−07 | 3.67647E−06 |
| Chloride | | | | |

TABLE 3

| COMPONENTS | mmol | mol |
|---|---|---|
| Aluminum Chloride 6H₂O | 2.249E−06 | 2.24896E−09 |
| Cadmium Chloride 2.5H₂O | 9.047E−05 | 9.04737E−08 |
| Rubidium Chloride | 5.24E−06 | 5.23967E−09 |
| Zirconium Chloride 8H₂O | 6.215E−06 | 6.21459E−09 |
| Cobalt Chloride 6H₂O | 1.824E−05 | 1.82437E−08 |
| Stannous Chloride 2H₂O | 4.487E−07 | 4.48673E−10 |
| Chromium Sulfate 15H₂O | 7.653E−07 | 7.65306E−10 |
| Nickelous Sulfate 6H₂O | 4.54E−07 | 4.53992E−10 |
| Sodium Flouride | 4.31E−05 | 4.30952E−08 |
| Cupric Sulfate 5H₂O | 1.81E−05 | 1.8096E−08 |
| Manganese Sulfate H₂O | 8.994E−07 | 8.99408E−10 |
| Ammonium Molybdate | 4.392E−06 | 4.39159E−09 |
| Germanium Dioxide | 2.305E−06 | 2.30476E−09 |
| Sodium Meta Vanadate | 4.59E−06 | 4.59016E−09 |
| Potassium Bromide | 4.555E−07 | 4.55462E−10 |
| Potassium Iodide | 5.012E−07 | 5.01205E−10 |
| Barium Acetate | 4.612E−06 | 4.61176E−09 |
| Silver Nitrate | 4.682E−07 | 4.68235E−10 |
| Titanium Tetrachloride ** | 1.368E−06 | 1.36842E−09 |
| Sodium Selenite | 9.079E−05 | 9.07861E−08 |
| Cupric Chloride 2H20 | 0.0002059 | 2.05882E−07 |
| Zinc Chloride | 0.0018382 | 1.83824E−06 |

The species of trace compounds listed in the tables are for illustration only. Alternative compositions, for example, salts with other counterions can be substituted without changing the trace compound added. Positive and negative trace compounds similarly can be combined in a single salt composition, for example cobalt bromide or fluoride might be substituted. Tables 1, 2 and 3 show preferred working ranges as well as favored concentrations for various components.

The present inventors have identified and investigated three extraordinary components, copper, zinc and nickel that greatly affect the growth of cells, especially PER.C6 cells in a base medium named Adenovirus Expression Medium (AEM) (Available from Invitrogen, Carlsbad, Calif.). Copper, nickel and zinc in combination were known by the inventor to increase bulk cell density, i.e., to result in greater growth. These ingredients (copper, nickel and zinc) thus far have not been exhaustively characterized. Zinc has now been evaluated separately and while it was found to improve overall cell growth the most notable effect is zinc's ability to provide consistent high density culturing of cells, for example, PER.C6 cells in AEM. Without additional zinc, AEM will not consistently support the passaging of cells if cultures reach day 4 densities greater than 2×10⁶ vc/mL (viable cells per milliliter). When cells are cultured to a density greater 2×10⁶ vc/mL, the subsequent subculture will fail to reach 1×10⁶ vc/mL. Subsequent subcultures will demonstrate similar lag if cell density is allowed to reach greater than 2×10⁶ vc/mL. The tables above show suggested concentrations. However, a total concentration may be used leaving out one or more compounds while increasing the others or one or more compounds such a copper or zinc might be omitted and those ions not replaced by other compounds.

In contrast, although copper and nickel in combination are shown to increase peak cell densities, the presence of each singly or in combination does not improve passaging of cells from high-density cultures.

In preliminary work the present inventors and coworkers noted that media, for example, AEM medium batches exhibited noted variability in peak cell densities. Specifically, the batches exhibiting low performance would not support cell growth of ≧2.0−×10⁶ cells/mL on day five post-planting. This low performance is deemed undesirably low for bioreactor protocols. Based on a hypothesis that there may be trace metals, which are not essential for cell growth, but their presence helps minimize the effects of trace metal contamination, and therefore their addition might lead to more consistent medium performance, additional trace elements were investigated as a possible means of mitigating or obviating the performance differences.

A trace element mix (TEM) was created and used in various test protocols. TEM contains copper, nickel, zinc, calcium and magnesium 10⁻³-10⁻⁴M. Preliminary observations collected from various concentrations of these metals yielded variable results improving some lots while having negative effects on others.

### EXAMPLE 1

The following media supplements were compared:
1) TEM.
2) TEM without zinc.
3) Zinc alone at the TEM concentration.
4) These supplements were each added to individual bottles of AEM at 1×, 3×, 5× and 10×. The conditions were then monitored by observing PER.C6 growth. Under conditions where 1×TEM was observed to increase cell growth toxic levels were not observed up to 10× TEM. Two of the components in TEM, Cu and Ni would individual be toxic at the 10× levels. These levels of trace elements would have been observed in the preliminary variable observations. Thus it was decided that total trace element concentrations were not responsible for the deleterious effects observed.

Conditions 1 and 3 (containing Zn) did not lag in the subsequent culture when passaged from overgrown day 5 cultures. In contrast Condition 2 did lag in the subsequent culture when passaged from overgrown day 5 culture.

### EXAMPLE 2

Copper and nickel have been previously reported as part of a group of components contributing to variability when supplemented to biomolecule production media such as AEM, and other chemically defined media such as used for 293 or HCO cells. Thus to further describe individual effects of manganese, cobalt, copper and nickel concentrations of these elements were added to AEM and used to monitor PER.C6 cell growth performance.

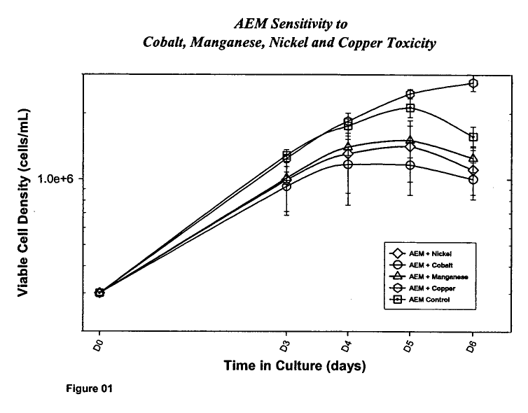

The elements were each tested at the following low concentrations: nickel (nitrate) at 0.004 and mg/L, cobalt (chloride) at 0.001 mg/L, manganese (chloride) at 0.028 mg/L and copper (chloride) at 0.013 mg/L. The results shown on FIG. 01 establish that copper produced the best results of all conditions tested. In contrast the other elements were inhibitory compared to control.

With the exception of the condition with (0.013 mg/L copper) all cultures apparently entered a lag growth phase after either day four or five. However, the culture containing 0.013 mg/L of copper continued in the log growth phase through day six. Thus, copper when added to AEM at 0.013 mg/L apparently extends log phase growth resulting in a higher cell count and cell mass. Cell mass correlates highly with production of biomolecules in cells engineered or adapted for such tasks.

Nickel when added to AEM at 0.004 mg/L inhibits PER.C6 cell growth.

Cobalt when added to AEM at 0.001 mg/L inhibits Per.C6 cell growth.

Manganese when added to AEM at 0.028 mg/L inhibits PER.C6 cell growth.

### EXAMPLE 3

A more robust trace element mix (TEM-2) was created and used in various test protocols. TEM-2 contains 22 trace elements at concentrations ranging from about 5×10⁻¹⁰ to about 10⁻⁷ M. The total concentration of adding trace components is about 6×10⁻⁷. Concentrations half this concentration and twice this concentration were effective though to differing degrees. Addition of this robust TEM-2 overcame the variability of the different batches that had been observed under the previous conditions but also generally improved cell growth and final density. Surprisingly. TEM-2 improved the poor performing batches more than the satisfactorily performing batches with the result that all batches were remarkedly consistent.

This experiment tests the ability of TEM-2 to eliminate the lot-to-lot variability in AEM batches. TEM-2 was added to a panel of eight batches of AEM representing high, medium and low performers.

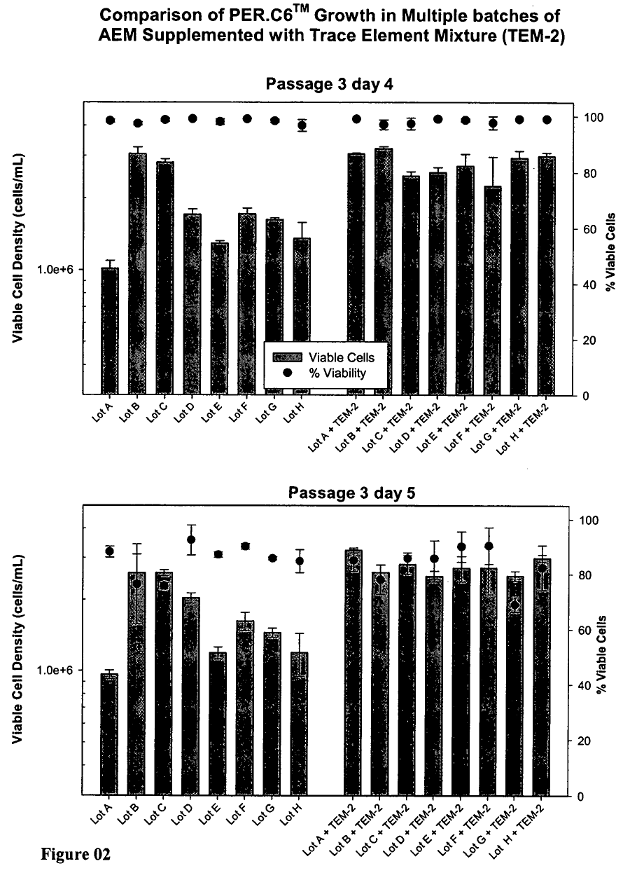

The PER.C6 (FIG. 02) cell growth performance in the eight AEM test batches (high, medium and low performance) was statistically equivalent after supplementation with TEM-2. The addition of TEM-2 eliminated the previously demonstrated batch-to-batch variability.

Thus surprisingly, addition of TEM-2 trace element solutions as a supplement to multiple lots of AEM has produced statistically equivalent PER.C6 cell growth performance regardless of the original performance of the medium for growing cells. Apparently a trace element balance is advantageous for overcoming batch-to-batch variability and providing batch-to-batch consistency. Addition of a trace element cocktail at rather high concentrations by traditional standards has been shown to minimize or alleviate variability between batches. This is especially surprising because some of the trace elements in this cocktail are added to a final concentration greater than a concentration observed for that trace element to cause serious deleterious or toxic effects individually, i.e., when the effects of the trace element are not balanced by opposing or protective trace elements such as a selection of trace elements in a cocktail of trace elements.

### EXAMPLE 4

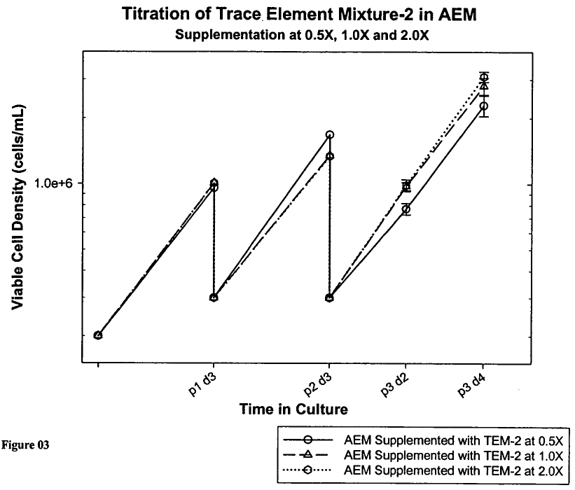

FIG. 03 shows that three different concentrations of TEM-2 demonstrated improved cell growth compared to control without. The 0.5× appeared to be slightly less an enhancer, than either the 1× or 2×, which were close in results to each other. But all concentrations showed improved results over control growth.

### EXAMPLE 5

This experiment was undertaken to demonstrate the ability of the supplement TEM-2 to prevent the adverse affects (example 2) caused by the addition of cobalt, nickel and manganese to AEM. The same concentrations as in example 2 were added to AEM which had been supplemented with TEM-2. They were then evaluated for PER.C6 cell growth.

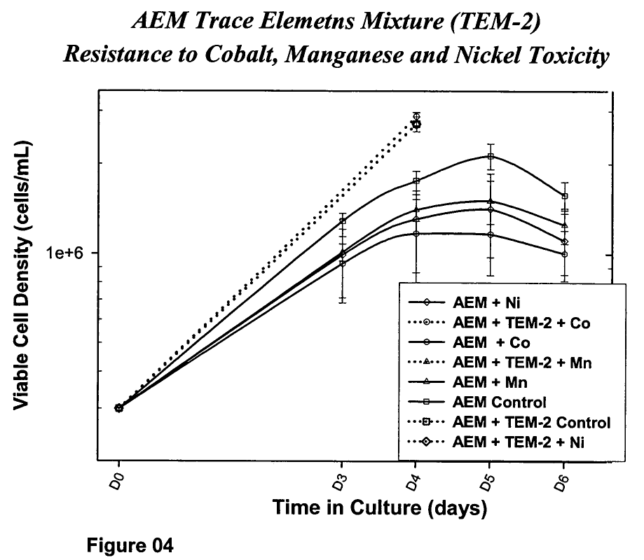

FIG. 04 shows that the addition of cobalt, nickel and manganese to AEM supplemented with TEM-2 had no effect on PER.C6 cell growth performance. Thus the addition of robust trace element solution (TEM-2) to prevented the toxicity caused by the individual trace metals. Surprisingly increasing the concentration of the trace compounds, some of which are considered toxins, ameliorated the toxic effects of trace components already present in the medium. Apparently concentrations are important, but a balance of trace compounds can mitigate detrimental effect of trace compounds. Thus rather than minimizing “toxic” components adding a trace element mix to establish a balance of several components spears to be a practical and economic way to produce media and to culture cells.

### EXAMPLE 6

This experiment was undertaken to demonstrate both the effects of supplementing AEM with TEM-3 and of adding the TEM-3 “supplement” during the formulation of AEM.

TEM-3 combines the benefits of TEM-2 (elimination of batch-to-batch variability and resistance to trace metal toxicity), the growth improving effects of copper and the high-density culturing ability of zinc.

All references to patents and publications discussed herein are hereby incorporated each in its entirety by reference. The inventor believes that such incorporation by reference is not necessary, in case a disclosure in any reference is deemed an essential teaching reserves the privilege and right to copy said disclosure to render more easily accessible said “essential” teaching. Inclusion of any reference does not indicate that the reference is or can be used as prior art to the present application. Such conclusions regarding prior art shall be based on patent law.

### EXAMPLE 7

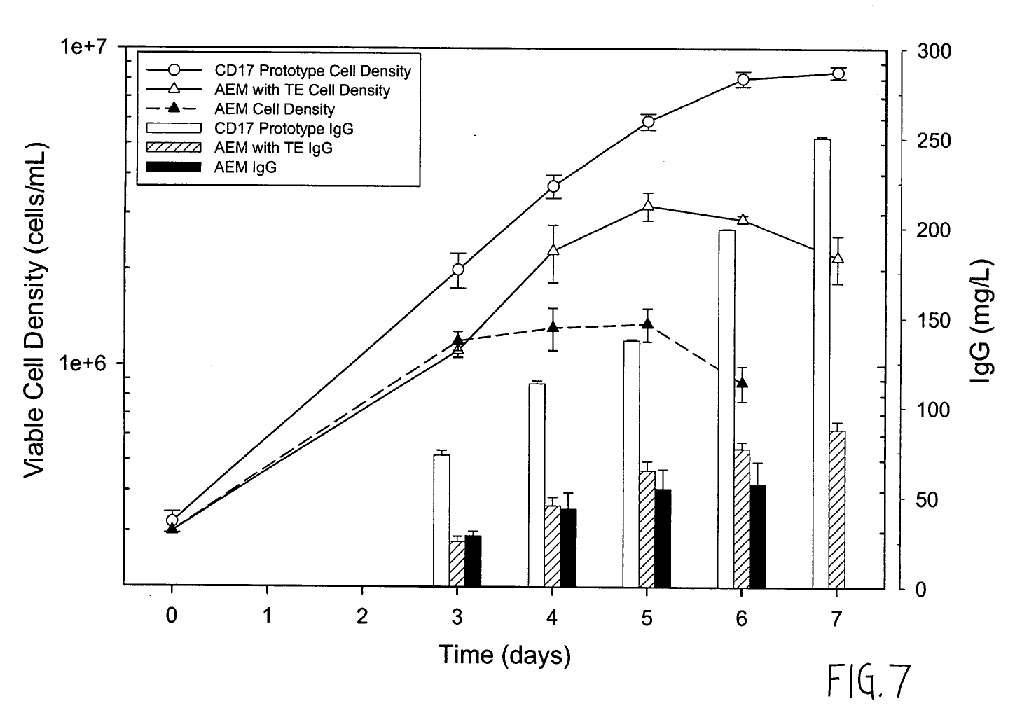

FIG. 07 shows surprisingly high titers of antibody production achievable with high cell densities made possible practicing the present invention. Similar results can be expected for 293 cells CHO cells and Per.C6 cells.

1. A composition for use in growing cells, said composition comprising trace compounds wherein a sum of concentrations of said trace compounds not including Zn is greater than or greater than: about 4×10⁻⁷M or not including copper about 4×10⁻⁶M.

2. The composition according to claim 1 wherein the trace compounds are selected from the group consisting of Sc, Ti, V, Cr, Mn, Fe, Co, Ni, Cu, Zn, Ga, Ge, As, Se, Br, Al, Si, P, Y, Zr, Nb, Mo, Tc, Ru, Rh, Rb, Ce, Ag, Pd, Ag, Cd, In, Sn, Sb, F, Te, Au, Pt, Bi, Ir, Os, Re, W, Ta and Hf.

3. The composition according to claim 1 wherein the trace compounds are selected from the group consisting of Al, Cd, Rb, Zr, Co, Sn, Cr, Ni, F, Cu, Mn, Mo, Ge, V, Br, I, Ba, Ag, Ti, Se, Cu and Zn.

4. The composition according to claim 2 comprising at least three of said trace compounds.

5. The composition according to claim 2 comprising at least five of said trace compounds.

6. The composition according to claim 2 comprising at least seven of said trace compounds.

7. The composition according to claim 3 comprising each of said trace compounds.

8. The composition according to claim 1 comprising an excess of zinc or copper, said excess of zinc or copper being at least or at least about 0.2, 0.5, 1.0. 1.5 or 2.0 times the sum of the concentrations of the other trace compounds.

9. The composition according to claim 1 made by adding trace compounds other than one of Ag or Ni in at least a five fold excess to the Ag or the Ni.

10. A composition comprising a cell selected from the group consisting of CD34⁺ hematopoietic cells and cells of myeloid lineage, 293 embryonic kidney cells, A-549, Jurkat, Namalwa, Hela, 293BHK cells, HeLa cervical epithelial cells and PER-C6 retinal cells, aka PER.C6, MDBK (NBL-1) cells, 911 cells, CRFK cells, MDCK cells, BeWo cells, Chang cells, Detroit 562 cells, HeLa 229 cells, HeLa S3 cells, Hep-2 cells, KB cells, LS 180 cells, LS 174T cells, NCI-H-548 cells, RPMI 2650 cells, SW-13 cells, T24 cells, WI-28 VA13, 2RA cells, WISH cells, BS-C-I cells, LLC-MK₂ cells, Clone M-3 cells, 1-10 cells, RAG cells, TCMK-1 cells, Y-1 cells, LLC-PK₁ cells, PK(15) cells, GH₁ cells, GH₃ cells, L2 cells, LLC-RC 256 cells, MH₁C₁ cells, XC cells, MDOK cells, VSW cells, TH-I, B1 cells, or derivatives thereof, fibroblast cells from any tissue or organ (including but not limited to heart, liver, kidney, colon, intestines, esophagus, stomach, neural tissue (brain, spinal cord), lung, vascular tissue (artery, vein, capillary), lymphoid tissue (lymph gland, adenoid, tonsil, bone marrow, and blood), spleen, fibroblast and fibroblast-like cell lines), TRG-2 cells, IMR-33 cells, Don cells, GHK-21 cells, citrullinemia cells, Dempsey cells, Detroit 551 cells, Detroit 510 cells, Detroit 525 cells, Detroit 529 cells, Detroit 532 cells, Detroit 539 cells, Detroit 548 cells, Detroit 573 cells, HEL 299 cells, IMR-90 cells, MRC-5 cells, WI-38 cells, WI-26 cells, MiCl₁ cells, CV-1 cells, COS-1 cells, COS-3 cells, COS-7 cells, Vero cells, DBS-FrhL-2 cells, BALB/3T3 cells, F9 cells, SV-T2 cells, M-MSV-BALB/3T3 cells, K-BALB cells, BLO-11 cells, NOR-10 cells, C₃H/IOTI/2 cells, HSDM₁C₃ cells, KLN205 cells, McCoy cells, Mouse L cells, Strain 2071 (Mouse L) cells, L-M strain (Mouse L) cells, L-MTK (Mouse L) cells, NCTC clones 2472 and 2555, SCC-PSA1 cells, NS0, NS1, Swiss/3T3 cells, Indian muntjac cells, SIRC cells, C_{II} cells, Jensen cells, COS cells and Sp2/0 cells, Mimic cells and/or derivatives thereof in serum-free culture;
zinc at a concentration is greater than or greater than about 0.0005, 0.001, 0.002, 0.004, 0.005, 0.007, 0.008, 0.01, 0.012, 0.015 or 0.02 mM; and
a cell culture medium that otherwise provides for the physical and chemical needs of the cell.

11. The composition according to claim 1 wherein the zinc concentration is about or is greater than about 0.5, 1, 2, 3, 4, 5, 7, 8, 10, 12, 15, 16, 18, 20, 22, 25 30, 35, 40, 45, 50, 60, 70, 80, 90 or 100×10⁻⁷M.

12. The composition according to claim 10 wherein the zinc concentration is about or is greater than about 0.5, 1, 2, 3, 4, 5, 7, 8, 10, 12, 15, 16, 18, 20, 22, 25 30, 35, 40, 45, 50, 60, 70, 80, 90 or 100×10⁻⁷M.

13. The composition according to claim 8, wherein the excess is not greater than or not greater than about 2, 5 or 10× the sum of the concentrations of the other trace compounds.

14. The composition according to claim 10 wherein the composition is insulin free.

15. The composition according to claim 2 comprising at least ten of said trace compounds.

16. The composition according to claim 2 comprising at least twelve of said trace compounds.

17. The composition according to claim 2 comprising at least sixteen of said trace compounds.

18. The composition according to claim 2 comprising at least twenty of said trace compounds.

19. The composition according to claim 1 made by adding trace compounds excluding Zn and Ni in at least a 5, 10, 20, 35, 30, 40, 50, 75, 80, 100, 150, 200, 300, 400, 500, 600, 700, 750, 800, 900, 1000, 1200, 1250, 1500, 1800, or 2000 fold excess to added Ni.

20. The composition according to claim 1 made by adding trace compounds excluding Zn and Cu in at least or at least about a 1.2, 1.5, 1.8, 2, 4, 5, or 10 fold excess to added Cu.

21. A composition for use in growing cells, said composition comprising trace compounds wherein a sum of concentrations of said trace compounds not including Zn is greater than about 10⁻⁷, 2×10⁻⁷, 3×10⁻⁷, 5×10⁻⁷, 7×10⁻⁷, 8×10⁻⁷, 9×10⁻⁷, 10×10⁻⁷ or 12×10⁻⁷M and Ni is not an added trace compound.

22. A method of improving cell culture medium comprising:
adding trace compounds to said medium to produce a decreased ratio of a transition metal selected from the group consisting of Ni, Cd, Hg and Pb to a total concentration of trace compounds.

23. The method according to claim 22 wherein the ratio is decreased by at least or at least about 2, 5, 10, 12, 15, 18, 20, 25, 40, 50, 60, 75, 80, 100, 150, 200, 500, or 1000.

24. The composition according to claim 1, wherein said sum does not exceed 10⁻⁶, 2×10⁻⁶, 3×10⁻⁶, 4×10⁻⁶, 5×10⁻⁶, 6×10⁻⁶, 7×10⁻⁶, 8×10⁻⁶, 9×10⁻⁶ or 10×10⁻⁶M.

25. A composition for use in growing cells, said composition comprising trace compounds wherein a sum of concentrations of said trace compounds not including Zn is less than or less than about 3×10⁻⁷M or less than or less than about 6×10⁻⁷M including Zn.

26. A method of growing cells in culture comprising providing a trace compound according to the present invention to a cell in a culture container and culturing said cell.

27. A method for inhibiting apoptosis of cells grown in culture comprising culturing said cells in a composition or in a presence of a medium according to the present invention.

28. A method of preventing or reducing lag in cells in culture comprising culturing cells in a composition or in the presence of a medium according to the present invention.

29. The method according to claim 28 wherein the cells are 293 cells or derivatives thereof.

30. A method of culturing cells to a density of at least or at least about 4×10⁶ comprising culturing said cells in a composition or in a medium according to the present invention.

31. The method according to claim 30 wherein said cells are cultured to at least or at least about 4×10⁶, 5×10⁶, 6×10⁶, 7×10⁶, 8×10⁶, 9×10⁶, or 10⁷ cells/ml.

32. The method according to claim 31 wherein said cells are selected from the group consisting of Per.C6, 293, hybridoma and Jurkat.

33. Method of producing protein in cells at a concentration about or at least about 1, 2, 4, 8, 10 or 12 mg/ml comprising culturing said cells according to a method of any one of claims 26-30.

34. A supplement that when added to a basal medium produces a medium according to the present invention.

35. A method of culturing cells in a base cell culture medium, the improvement being adding trace compounds to said medium to reduce a ratio of a metal selected from the group consisting of Ni, Cd and Pb to total trace compounds by about or about at least 200, 500, 800, 100, 1200, 1500, 2000, or 2500 times.

36. The composition according to claim 1 further comprising cells selected from the group consisting of PER.C6 cells, 293 cells and HeLa cells.

37. The composition according to claim 36 comprising 293 cells.

38. The composition according to claim 36 comprising PER.C6 cells.

39. The composition according to claim 10, wherein said cells are cultured to a concentration selected from the group consisting of at least or at least about 1.8×10⁶, 2.0×10⁶, 3×10⁶, 4×10⁶, 5×10⁶, 6×10⁶, 7×10⁶, 8×10⁶ and 9×10⁶ cells per ml.

40. A composition of cells cultured to a concentration of at least 10⁷ said cells selected from the group consisting of CD34⁺ hematopoietic cells and cells of myeloid lineage, 293 embryonic kidney cells, A-549, Jurkat, Namalwa, Hela, 293, BHK cells, HeLa cervical epithelial cells and PER-C6 retinal cells, aka PER.C6, MDBK (NBL-1) cells, 911 cells, CRFK cells, MDCK cells, BeWo cells, Chang cells, Detroit 562 cells, HeLa 229 cells, HeLa S3 cells, Hep-2 cells, KB cells, LS 180 cells, LS 174T cells, NCI-H-548 cells, RPMI 2650 cells, SW-13 cells, T24 cells, WI-28 VA13, 2RA cells, WISH cells, BS-C-I cells, LLC-MK₂ cells, Clone M-3 cells, 1-10 cells, RAG cells, TCMK-1 cells, Y-1 cells, LLC-PK₁ cells, PK(15) cells, GH₁ cells, GH₃ cells, L2 cells, LLC-RC 256 cells, MH₁C₁ cells, XC cells, MDOK cells, VSW cells, TH-I, B1 cells, or derivatives thereof, fibroblast cells from any tissue or organ (including but not limited to heart, liver, kidney, colon, intestines, esophagus, stomach, neural tissue (brain, spinal cord), lung, vascular tissue (artery, vein, capillary), lymphoid tissue (lymph gland, adenoid, tonsil, bone marrow, and blood), spleen, fibroblast and fibroblast-like cell lines), TRG-2 cells, IMR-33 cells, Don cells, GHK-21 cells, citrullinemia cells, Dempsey cells, Detroit 551 cells, Detroit 510 cells, Detroit 525 cells, Detroit 529 cells, Detroit 532 cells, Detroit 539 cells, Detroit 548 cells, Detroit 573 cells, HEL 299 cells, IMR-90 cells, MRC-5 cells, WI-38 cells, WI-26 cells, MiCl₁ cells, CV-1 cells, COS-1 cells, COS-3 cells, COS-7 cells, Vero cells, DBS-FrhL-2 cells, BALB/3T3 cells, F9 cells, SV-T2 cells, M-MSV-BALB/3T3 cells, K-BALB cells, BLO-11 cells, NS0, NS1, NOR-10 cells, C₃H/IOTI/2 cells, HSDM₁C₃ cells, KLN205 cells, McCoy cells, Mouse L cells, Strain 2071 (Mouse L) cells, L-M strain (Mouse L) cells, L-MTK (Mouse L) cells, NCTC clones 2472 and 2555, SCC-PSA1 cells, Swiss/3T3 cells, Indian muntjac cells, SIRC cells, C_{II} cells, Jensen cells, COS cells and Sp2/0 cells, Mimic cells, and/or derivatives thereof in serum-free culture.

41. A Method of producing a biomolecule, virus particle or virus of interest comprising culturing a cell engineered to produce said biomolecule of interest in a composition according to the present invention, said molecule produced in said culture at a concentration exceeding 4, 5, 6, 7, 8, 9, 10 11, 12 14, 15, 18, 20 or 25×10⁻⁴ g/ml or about 3, 4 or 5 mg/ml.

42. A method of reducing variability of cell growth and/or bioproduction in cell culture comprising providing a culture medium with a supplement according to claim 34 and culturing cells in same.

43. The method according to claim 42 comprising adding said supplement to said culture medium before said culture medium is contacted with said cells.

44. The method according to claim 42 comprising adding said supplement to said culture medium while said culture medium is in contact with said cells.

45. The composition according to claim 1, wherein a concentration of each trace compound present in said composition is selected from the values or ranges from table 1.

46. The composition according to claim 45 wherein the composition comprises Zn.

47. The composition according to claim 45 wherein the composition comprises Cu.

48. The composition according to claim 45 wherein the composition comprises Zn and Cu.

46. The composition according to claim 45 wherein the composition comprises all compounds of the table except at least one compound selected from the group consisting of Zn and Cu.

47. The composition According to claim 1 further comprising CHO cells or myeloma cells in culture and Zn and/or copper at a concentration of at maximum or at maximum about 1.84×10⁻⁷, more preferably, 4.6×10⁻⁷, and still more preferably 9.2×10⁻⁷, for zinc and 2.05×10⁻⁸, more preferably 5.15×10⁻⁸, and still more preferably 1.03×10⁻⁷, for copper.

48. The composition according to claim 1 further comprising CHO cells or myeloma cells in culture and Zn and/or copper at a concentration of at least or at least about 1.84×10⁻⁵, more preferably, 7.4×10⁻⁶, and still more preferably 3.7×10⁻⁶, for zinc and 2.05×10⁻⁶, more preferably 8.2×10⁻⁷, and still more preferably 4.1×10⁻⁷, for copper.

49. A method for decreasing detrimental effect of trace components comprising adding additional trace components.

50. The method according to claim 49 wherein the additional trace components added include at least one trace component already present in a cell culture medium.

51. The method according to claim 49 wherein the additional trace components added include at least one trace component not present in a cell culture medium.

52. The method according to one of claims 50 or 51 wherein the at least one trace component includes at least one selected from the group consisting of Al, Cd, Rb, Zr, Co, Sn, Cr, Ni, F, Cu, Mn, Mo, Ge, V, Br, I, Ba, Ag, Ti, Se, Cu and Zn.

53. The method according to claim 52 wherein the at least one trace component includes at least one selected from the group consisting of Cu and Zn.

54. The method according to claim 49, wherein the trace components whose detrimental effect is to be decreased were not intentionally present.

55. The method according to claim 49, wherein the trace components whose detrimental effect is to be decreased were present as a byproduct of at least one ingredient intentionally added.

56. The method according to claim 49, wherein the trace components whose detrimental effect is to be decreased were present as a result of manufacturing of handling a cell cure medium.

57. A method for reducing batch-to-batch variability in cell culture comprising adding additional trace components.

58. The method according to claim 57 wherein the additional trace components added include at least one trace component already present in a cell culture medium.

59. The method according to claim 57 wherein the additional trace components added include at least one trace component not present in a cell culture medium.

60. The method according to one of claims 58 or 59 wherein the at least one trace component includes at least one selected from the group consisting of Al, Cd, Rb, Zr, Co, Sn, Cr, Ni, F, Cu, Mn, Mo, Ge, V, Br, I, Ba, Ag, Ti, Se, Cu and Zn.

61. The method according to claim 60 wherein the at least one trace component includes at least one selected from the group consisting of Cu and Zn.

62. The method according to claim 57, wherein the trace components whose detrimental effect is to be decreased were not intentionally present.

63. The method according to claim 57, wherein the trace components whose detrimental effect is to be decreased were present as a byproduct of at least one ingredient intentionally added.

64. The method according to claim 57, wherein the trace components whose detrimental effect is to be decreased were present as a result of manufacturing of handling a cell cure medium.

65. The composition according to claim 1, wherein Fe is not included in the sum of concentrations of trace elements.
